(19) United States
(12) Patent Application Publication
(10) Pub. No.: US 2015/0268226 A1
(43) Pub. Date: Sep. 24, 2015

(54) MULTIMODAL MICROSCOPY FOR AUTOMATED HISTOLOGIC ANALYSIS OF PROSTATE CANCER

(71) Applicants: Rohit Bhargava, Urbana, IL (US); Saurabh Sinha, Champaign, IL (US); Jin Tae Kwak, Champaign, IL (US)

(72) Inventors: Rohit Bhargava, Urbana, IL (US); Saurabh Sinha, Champaign, IL (US); Jin Tae Kwak, Champaign, IL (US)

(73) Assignee: The Board of Trustees of the University of Illinois

(21) Appl. No.: 13/090,384

(22) Filed: Apr. 20, 2011

Related U.S. Application Data
(60) Provisional application No. 61/326,151, filed on Apr. 20, 2010.

(51) Int. Cl. G01N 33/50; G01N 21/55; G06F 19/00

(52) U.S. Cl. CPC G01N 33/5091; G06F 19/345; G01N 21/55; G01N 2800/342

(57) ABSTRACT

The present disclosure relates to methods of diagnosing prostate cancer using different imaging methods. For example, it is shown herein that combining a Fourier transform infrared (FT-IR) spectroscopic image with an optical image (such as a hematoxylin and eosin image) allows for automated detection of prostate cancer with high accuracy.

### CROSS-REFERENCE TO RELATED APPLICATION

This application claims priority to U.S. Provisional Application No. 61/326,151 filed Apr. 20, 2010, herein incorporated by reference in its entirety.

### ACKNOWLEDGMENT OF GOVERNMENT SUPPORT

This invention was made with government support under grant number W81XWH-07-01-0242 awarded by The Department of Defense and grant number R01CA138882 from the National Cancer Institute. The government has certain rights in the invention. The project is also supported by National Institutes of Health grant number R01CA138882.

### FIELD

This application relates to methods of diagnosing prostate cancer, for example using light microscopy and fourier transform infared spectroscopic imaging.

### BACKGROUND

Prostate cancer (PCa) is the single most prevalent cancer in US men, accounting for one-third of non-skin cancer diagnoses every year [1]. Screening for the disease is widespread and for almost a million cases a year [2-4], a biopsy is conducted to detect or rule out cancer [5]. Manually-conducted histologic assessment of tissue upon biopsy forms the definitive diagnosis of PCa [6]. This need places a large demand on pathology services and manual examination limits speed and throughput. Alternative methods for histologic recognition can greatly aid in alleviating workloads, assuring quality control and reducing costs.

Since the tissue does not have appreciable contrast in optical brightfield microscopy (FIG. 1A), samples are commonly stained using hematoxylin and eosin (H&E) prior to review by a pathologist. The stain is specific in limited terms—staining protein-rich regions pink and nucleic acid rich regions of the tissue blue (FIG. 1B). A pathologist is trained to recognize, from a stained tissue sample, the morphology of specific cell types and their structural alterations that indicate disease. In prostatic carcinoma, which comprises more than 95% of prostate cancers, the cells of interest are epithelial cells. Epithelial cells line 3D ducts in intact tissue and, hence, appear as cells lining empty circular regions (lumens) in images of histologic sections. Patterns of distortions of lumen appearance and spacing, as well as the arrangement of epithelial cells relative to lumens, have been characterized to indicate cancer and characterize its severity (Gleason grade) [7, 8]. The greater the distortion and loss of regular structure, the worse (higher grade) the cancer.

Recognizing structural distortions indicative of disease is a manual pattern recognition process that matches patterns in the sample to standard patterns. Manual examination is powerful in that humans can recognize disease from a wide spectrum of normal and disease states, can overcome confounding artifacts, detect unusual cases and even recognize deficiencies in diagnoses. Manual examination, unfortunately, is time-consuming and leads routinely to variability in grading disease [7]. Computer-aided recognition of disease samples and Gleason grade patterns, hence, holds the potential for more accurate, reproducible and automated diagnoses. Unfortunately, tissue samples stain variably in populations due to biological diversity, with variations in stain composition, processing conditions and histotechnologists. The net result confounds automated image analysis and human-competitive recognition of cancer has not been automated for routine use. A robust means of automatically detecting epithelium and correlating its spatial patterns to determining cancer presence is highly desirable but yet unsolved.

Several efforts have been made to develop automated systems for the diagnosis and grading of microscopic prostate images. These include methods to identify distinct tissue compositions [9, 10] as well as several methods for automatic grading [11-20]. The majority of these methods have extracted texture and/or morphological features to characterize tissue samples. Histologic objects such as nuclei, lumen, or gland have been mainly used to extract morphological features [11, 12, 16-19]. Fourier Transform [13], Wavelet Transform [14, 15, 19], and Fractal Analysis [19, 20] have been the techniques commonly used to obtain texture features. In addition to these features, color [19] and graph-based [17] features have also been used. A number of classifiers have been tested on various features and data sets, although the choice of classifiers seems to have been less significant than the feature extraction step [19, 20].

Despite these lines of progress in automated diagnosis, an important concern is that the varying properties of images, due to acquisition settings [15, 21] and staining [22], may affect the classification results substantially. Although the issue of image variation by different acquisition settings has been addressed [15, 21], no method has been validated across data sets under different staining conditions.

A major roadblock has been the limited information present in the data. For example, different cell types and morphologies need to be distinguished based entirely on differences in color between regions. Immunohistochemical probes add useful information to diagnostic processes and are effective in understanding specific aspects of the disease, e.g. loss of basement membrane. For routine diagnostic pathology, however, the use of such molecular stains is expensive, time-consuming and does not actually address the need for an operator-free method. Additional molecular data is now available using label-free spectroscopic imaging, also known as chemical imaging.

Prostatic epithelial cells (and other cell types) [23] have recently been automatically recognized using a novel form of chemical imaging based on mid-infrared (IR) spectroscopy. Fourier transform infrared (FT-IR) spectroscopic imaging provides non-perturbing imaging by combining the spatial specificity of optical microscopy with the molecular selectivity of vibrational spectroscopy. Mid-IR spectral frequencies are resonant with the fundamental vibrational mode frequencies in molecules; hence, the IR absorption spectrum at each pixel is a quantitative record of composition [24]. The spectral patterns of different cell types being different, computerized pattern recognition can be used to assign each pixel into constituent cell types. The final result of recording data and mathematical analysis is images of tissue that are color coded for cell type. The process is illustrated in FIG. 2. The approach has been used by a number of groups and is summarized in recent edited volumes [25, 26]. Since the numerical algorithms are automated, quantification of accuracy and statistical confidence in results is facile [27].

### SUMMARY

Provided herein are methods which combine two techniques (optical microscopy following H&E staining, and FT-IR imaging), and provide higher accuracy diagnoses that cannot be achieved using H&E images alone. This new and automated method can classify cancer versus non-cancer prostate tissue samples. The classification algorithm uses morphological features (such as geometric properties of epithelial cells/nuclei and lumens) that are quantified based on H&E stained images as well as FT-IR images of the samples. By restricting the features used to geometric measures, the method mimics the pattern recognition process employed by human experts, to achieve a robust classification procedure that produces consistently high accuracy across independent data sets.

The present application provides methods of diagnosing prostate cancer. For example, the method can include overlapping (or registering) a Fourier transform infrared (FT-IR) spectroscopic image of a first prostate sample with a hematoxylin and eosin image of a second prostate sample, thereby generating an overlapped (or registered) image. Epithelial cells in the overlapped image are then identified, as well as nuclei and lumens in the epithelial cells. Once these features are identified, the method includes extracting and classifying features from the nuclei and lumens (and in some examples also features from epithelium) in the overlapped image and analyzing the extracted and classified features from the nuclei and lumens (and in some examples also features from epithelium, such as the size of the epithelial cells) for prostate cancer. For example, if smaller lumens and an increase in the number of nuclei relative to a normal prostate control sample are detected, this indicates that the prostate sample is positive for prostate cancer. In contrast, if similar lumens and a similar number of nuclei relative to a normal prostate control sample are detected, this indicates that the prostate sample is negative for prostate cancer.

Also provided herein are computer-readable storage media having instructions thereon for performing a method of diagnosing cancer. Such media can include instructions describing methods for acquiring a Fourier transform infrared (FT-IR) spectroscopic image of a first sample and a H&E image of a second sample; overlaying the H&E image with the FT-IR image; detecting nuclei and lumen in the overlaid image; extracting and classifying features of the detected nuclei and lumen; and analyzing the extracted and classified features for cancer.

The foregoing and other objects and features of the disclosure will become more apparent from the following detailed description, which proceeds with reference to the accompanying figures.

### BRIEF DESCRIPTION OF THE DRAWINGS

FIGS. 1A-1C. Staining allows visualization of tissue features. (a) an unstained image has little contrast while (b) the application of H&E stain highlights nucleic acid-rich regions as blue and protein-rich regions at pink. (c) structure of a prostate gland. The stain is universal in that it is not diagnostic of cell type or disease. The stain serves only to provide contrast that is subsequently used by a human to recognize cell types and diagnose disease.

FIGS. 2A-2E. IR imaging data and its use in histologic classification. (Upper row) IR imaging data (b) is acquired for an unstained tissue section (a). The data is then classified into cell types and a classified image (c) is obtained. The colors indicate cell types in a histologic model of prostate tissue. This method is robust and applied to hundreds of tissue samples using the tissue microarray (TMA) format. (Lower row) H&E (d) and IR classified (e) images of a part of the TMAs used.

FIGS. 3A-3E. Overview of System. (a, b) FTIR spectroscopic imaging data-based cell-type classification (IR classified image), is overlaid with H&E stained image (a), leading to segmentation of nuclei and lumens in a tissue sample (b). (c, d, e) Features are extracted and selected (c), and used by the classifier (d) to predict (e) whether the sample is cancerous or benign.

FIG. 4. Image Registration. H&E stained images and IR classified images are first converted into binary images. The IR classified image is overlaid with the H&E stained image by affine transformation, with the optimal matching being found by minimizing the absolute intensity difference between two images. After registration, original annotations (color and/or cell-type information) of each image are restored.

FIG. 5. Nucleus Detection. Smoothing and adaptive histogram equalization are performed to alleviate variability in H&E stained image and to obtain better contrast. “RG−B” conversion followed by thresholding characterizes the areas where nuclei exist. Morphological closing operation is performed to fill holes and gaps within nuclei, and a watershed algorithm segments each individual nuclei. The segmented nuclei are constrained by their shape, size, and average intensity and epithelial cell classification (green pixels) provided by the overlaid IR image.

FIG. 6. Exemplary Features. Each panel shows one example feature, along with the distributions of the feature's values for cancer (solid line) and benign (dashed line) classes.

FIG. 7. Global and Local Feature Extraction. Global features are extracted from the entire tissue sample, and local features are extracted by sliding a window of a fixed size across the tissue sample and computing summary statistics, such as standard deviation, of window-specific scores. In this example, the global feature “number of nuclei” has value 755, while one example position of the sliding window is shown, with “number of nuclei”=29.

FIG. 8. Importance of 17 feature categories. The average “maximal relevance” of features belonging to each feature category is shown, for both data sets, sorted in decreasing order for the first data set.

FIG. 9. List of features and their maximal relevance and “mRMR rank”. In the second column, G and L represent global and local features, respectively. AVG, STD, TOT, and MAX denote the average, standard deviation, total amount, and extremal value of features. * In computing local features representing “size of lumen”, two options are available: one is to consider only the part of the lumen within the window, and the other is to consider the entire lumen into account. Asterisk indicates that the former option was chosen.

FIG. 10. Optimal features for distinguishing cancer and benign tissue samples. The four features shown here are always present in the optimal feature set chosen by the classifier.

FIGS. 11A and 11B are graphs showing the importance of 17 feature categories across cross-validation. “Maximal relevance” for both datasets (a) Data1 (b) Data2 is consistent over all folds of cross-validation

FIGS. 12A and 12B are graphs showing the frequency of optimal features across cross-validation. The features in the optimal feature set are relatively constant for both datasets (a) Data1 (b) Data2 over all folds of cross-validation.

FIGS. 13-16 represent flowcharts of methods that can be used to implement embodiments described herein.

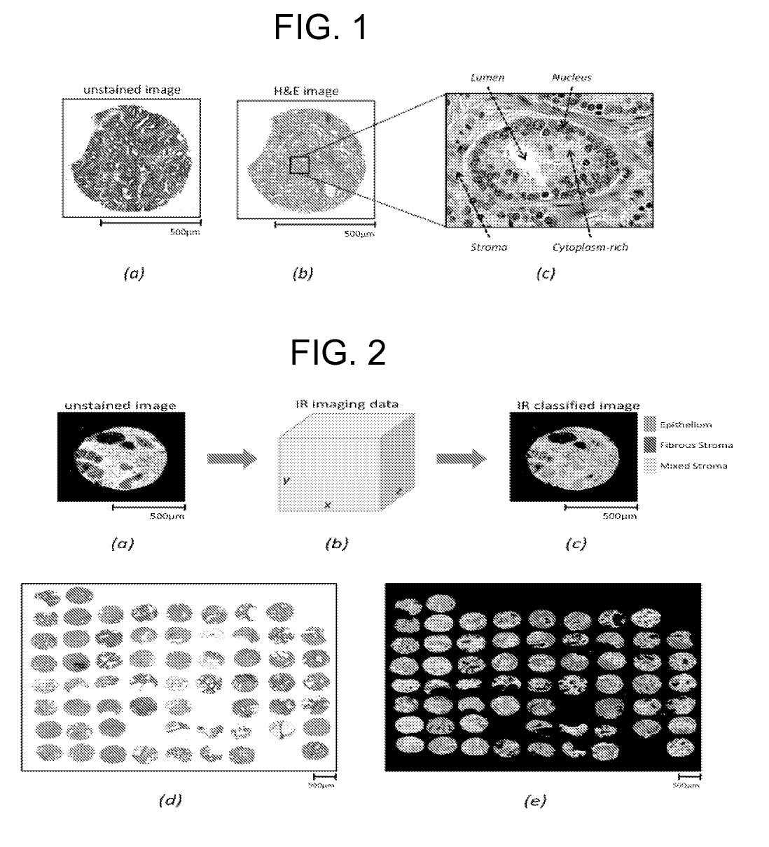

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including”; hence, “comprising A or B” means “including A” or “including B” or “including A and B.”

Cancer: Malignant neoplasm, for example one that has undergone characteristic anaplasia with loss of differentiation, increased rate of growth, invasion of surrounding tissue, and is capable of metastasis.

Control: A “control” refers to a sample or standard used for comparison with an experimental or test sample. In some embodiments, the control is a sample obtained from a healthy patient (such as a healthy or non-cancerous prostate sample) or a non-tumor tissue sample obtained from a patient diagnosed with prostate cancer. In some embodiments, the control is a historical control or standard reference value or range of values (such as a previously tested control sample, a group of samples that represent the average lumen characteristics or number of nuclei in prostate cancer tissue or normal prostate tissue).

Diagnose: The process of identifying a medical condition or disease, for example from the results of one or more diagnostic procedures. In particular examples, diagnosis includes determining the prognosis of a subject, such as determining the likely outcome of a subject having a disease (e.g., prostate cancer) in the absence of additional therapy (e.g., life expectancy), for example predicting the likely recurrence of prostate cancer in a human subject after prostatectomy.

Normal cells or tissue: Non-tumor, non-malignant cells and tissue.

Prostate Cancer: A malignant tumor, generally of glandular origin, of the prostate. Prostate cancers include adenocarcinomas and small cell carcinomas. Many prostate cancers express prostate specific antigen (PSA).

Subject: Includes any multi-cellular vertebrate organism, such as human and non-human mammals (e.g., veterinary subjects). In some examples, a subject is one who has cancer, or is suspected of having cancer, such as prostate cancer.

Suitable methods and materials for the practice and/or testing of embodiments of the disclosure are described below. Such methods and materials are illustrative only and are not intended to be limiting. Other methods and materials similar or equivalent to those described herein also can be used. For example, conventional methods well known in the art to which a disclosed invention pertains are described in various general and more specific references.

### Methods of Diagnosing Prostate Cancer

The present application provides methods for diagnosing prostate cancer. In some examples, subjects suspected of having or known to have prostate cancer are selected, and a prostate sample obtained (such as a biopsy sample). In some examples, if the sample is determined to be positive for prostate cancer, the subject is selected for treatment of the prostate cancer, such as surgical resection of the cancer or prostate; radiation therapy, or chemotherapy, or combinations thereof. Such treatments are known in the art.

In particular examples the method includes overlapping a Fourier transform infrared (FT-IR) spectroscopic image of a first prostate sample with a hematoxylin and eosin (H&E) image of a second prostate sample. This process is also referred to as image registration. Methods of processing a prostate sample for FT-IR analysis and H&E staining are routine in the art. In some examples, the FT-IR image is obtained from an unstained sample. The first and second prostate samples can be the same sample, for example where the FT-IR image is obtained, then the sample stained with H&E, and an optical microscopy (e.g., light microscopy) image obtained. In other examples, the first prostate cancer sample and the second prostate sample are different sections of the same sample, such as serial or adjacent tissue sections.

Epithelial cells present in the resulting overlapped image are then identified. For example, prostatic epithelial cells can be automatically recognized using chemical imaging based on mid-infrared (IR) spectroscopy. Fourier transform infrared (FT-IR) spectroscopic imaging provides non-perturbing imaging by combining the spatial specificity of optical microscopy with the molecular selectivity of vibrational spectroscopy.

Once the epithelial cells are identified, nuclei and lumens in the epithelial cells are identified in the overlapped image. Methods for identifying such structures are provided herein. Features from the nuclei and lumens in the overlapped image are then extracted and classified. H&E-staining enhances the segmentation of nuclei and lumens. The cellular and nuclear morphology of epithelial nuclei and lumens are different in normal and cancerous tissues, and thus can be used to diagnose prostate cancer.

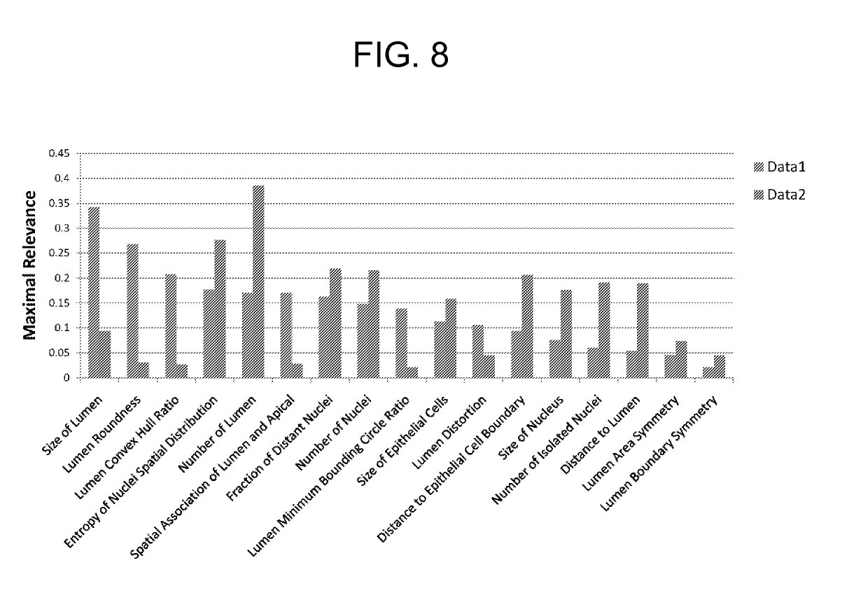

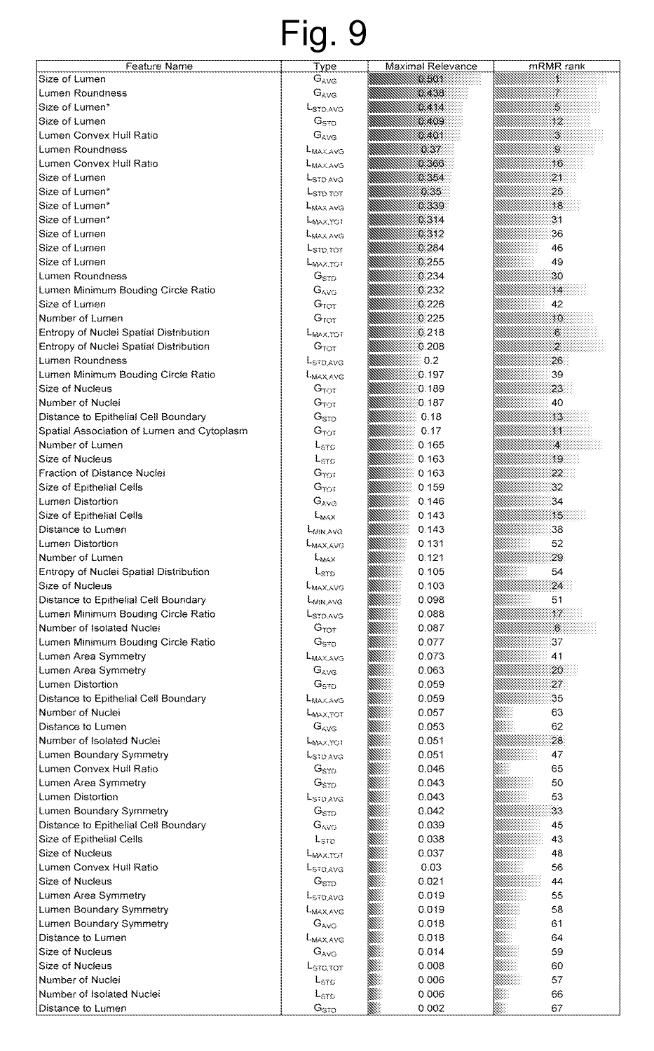

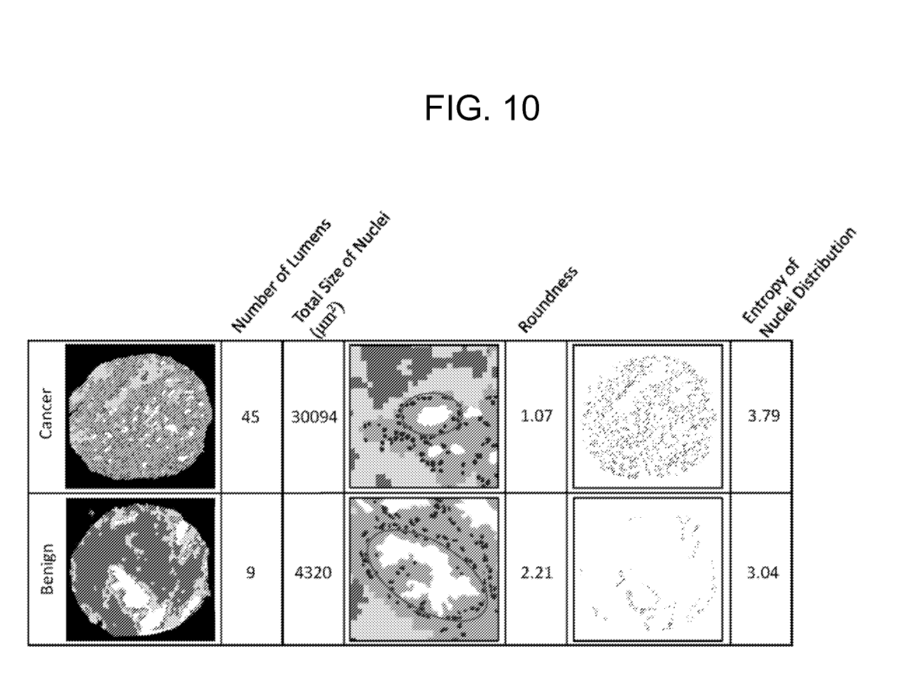

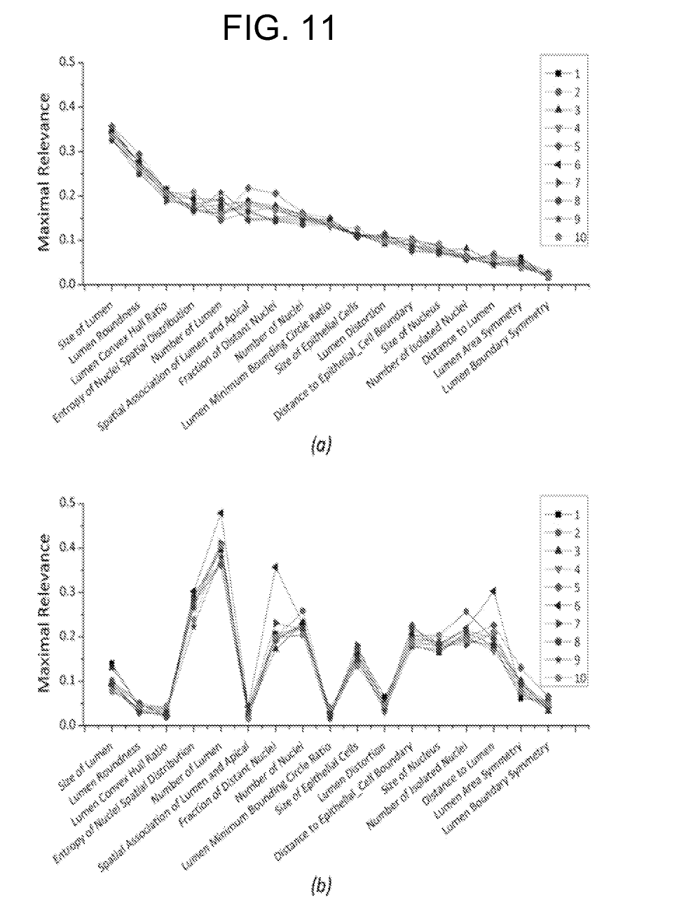

Exemplary features for nuclei and lumens are provided in FIG. 9. FIG. 9 lists 67 features that can be extracted and classified. However, one skilled in the art will appreciate that fewer or additional features may be used. For example, at least 4, at least 5, at least 10, at least 12, at least 15, at least 16, at least 17, at least 20, at least 25, at least 30, at least 35, at least 40, at least 45, at least 50, at least 55, at least 60, at least 65 or all of the features in FIG. 9 can be used. In some examples, one or more features for nuclei and lumens provided in FIG. 8 or 11 are used, such as at least 4, at least 5, at least 6, at least 7, at least 8, at least 9, at least 10, at least 11, at least 12, at least 13, at least 14, at least 15, at least 16, or all of the features in FIG. 8 or 11 can be used. In one example, all four features shown in FIG. 10 are used.

The method further includes analyzing the extracted and classified features from the nuclei and lumens for prostate cancer. In H&E stained images, lumens are recognized to be empty white spaces surrounded by epithelial cells. Patterns of distortions of lumen appearance and spacing, as well as the arrangement of epithelial cells relative to lumens, can be characterized to indicate prostate cancer and characterize its severity. In normal tissues, lumens are larger in diameter and can have a variety of shapes. In cancerous tissues, lumens are progressively smaller with increasing grade and generally have less distorted elliptical or circular shapes. The greater the distortion and loss of regular structure of the lumen, the worse (higher grade) the cancer. In addition, the number of nuclei differs between normal and cancerous tissues, with cancerous tissues having more. Thus, if smaller lumens and an increase in the number of nuclei are detected relative to a normal prostate control sample, this indicates that the prostate sample is positive for prostate cancer. In contrast, if similar lumens and a similar number of nuclei relative to a normal prostate control sample are detected, this indicates that the prostate sample is negative for prostate cancer, thereby diagnosing prostate cancer.

In some examples, one or more of the method steps used in diagnosing prostate cancer are performed on a suitably programmed computer. In some examples, the computer provides an output indicating whether the test prostate sample is cancerous or not. In other examples, the extracted and classified features are manually analyzed.

In some examples, an increase of at least 25%, at least 50%, at least 75%, or at least 90% in the number of nuclei in the test prostate sample relative to a normal prostate control sample indicates that the test prostate sample is positive for prostate cancer. In contrast, if there are a similar number of nuclei (e.g., +/−<5%, +/−<1%, such as less than a 5% difference, less than 4%, less than 3%, less than 2%, less than 1%, or less than a 0.5% difference) in the test prostate sample relative to the normal prostate control sample this indicates that the test prostate sample is negative for prostate cancer.

In some examples, a decrease of at least 25%, at least 50%, at least 75%, or at least 90% in the lumen volume in the prostate sample relative to a normal prostate control sample indicates that the prostate sample is positive for prostate cancer. In contrast, if the lumen volume is similar (e.g., +/−<5%, +/−<1%, such as less than a 5% difference, less than 4%, less than 3%, less than 2%, less than 1%, or less than a 0.5% difference) in the prostate sample relative to the normal prostate control sample this indicates that the prostate sample is negative for prostate cancer.

In some examples the method also includes acquiring the FT-IR spectroscopic image and the hematoxylin and eosin image. The method can also include preparing the test prostate samples for such imaging using routine methods.

In particular examples, the methods provided herein have a sensitivity of at least 90%, at least 95%, at least 98%, or at least 99% sensitivity, wherein sensitivity is the probability that a statistical test will be positive for a true statistic. In particular examples, the methods provided herein have a specificity of at least 90%, at least 95%, at least 98%, or at least 99% specificity, wherein specificity is the probability that a statistical test will be negative for a negative statistic.

### Biological Samples

Disclosed methods can be performed using biological samples obtained from any subject having or suspected of having prostate cancer. Such samples can be referred to as test samples. A typical subject is a human male; however, any mammal that has a prostate that may develop cancer can serve as a source of a biological sample useful in a disclosed method. Exemplary biological samples useful in a disclosed method include tissue samples (such as, prostate biopsies and/or prostatectomy tissues) or prostate cell samples (such as can be collected by prostate massage, in the urine, or in fine needle aspirates). Samples may be fresh or processed post-collection (e.g., for archiving purposes). In some examples, processed samples may be fixed (e.g., formalin-fixed) and/or wax- (e.g., paraffin-) embedded. Fixatives for mounted cell and tissue preparations are well known in the art and include, without limitation, 95% alcoholic Bouin's fixative; 95% alcohol fixative; B5 fixative, Bouin's fixative, formalin fixative, Karnovsky's fixative (glutaraldehyde), Hartman's fixative, Hollande's fixative, Orth's solution (dichromate fixative), and Zenker's fixative (see, e.g., Carson, Histotechology: A Self-Instructional Text, Chicago: ASCP Press, 1997).

In some examples, the sample (or a fraction thereof) is present on a solid support. Solid supports useful in a disclosed method need only bear the biological sample and, optionally, but advantageously, permit the convenient detection of components (e.g., lumens, nuclei, epithelial cells) in the sample. Exemplary supports include microscope slides (e.g., glass microscope slides or plastic microscope slides), coverslips (e.g., glass coverslips or plastic coverslips), tissue culture dishes, multi-well plates, membranes (e.g., nitrocellulose or polyvinylidene fluoride (PVDF)) or BIACORE™ chips.

### Control Samples

In some methods, the experimental sample is measured relative to a standard value or a control sample. Standard values can include, without limitation, the average lumen characteristics or number of nuclei (or range of values) in a normal prostate (e.g., calculated in an analogous manner to the prostate cancer sample) or the average lumen characteristics or number of nuclei (or range of values) in a prostate sample obtained from a patient or patient population in which it is known that prostate cancer was present. For example, standard values can include, without limitation, the average characteristics for those features listed in any of FIGS. 8-10 (such as the 67 features in FIG. 9) in a normal prostate or in a prostate sample obtained from a patient or patient population in which it is known that prostate cancer was present. The values for the features in the control are calculated in an analogous manner to the test prostate cancer sample. A control sample can include, for example, normal prostate tissue or cells, prostate tissue or cells collected from a patient or patient population in which it is known that prostate cancer was not present, or prostate tissue or cells collected from a patient or patient population in which it is known that prostate cancer was present.

An increase in the number of nuclei relative to a normal sample may mean, for example, that the number of nuclei in the test sample is at least at least 15%, at least 20%, at least 25%, at least 30%, at least 50%, at least 75%, at least 100%, at least 150%, or at least 200% higher, of the normal (non-prostate cancer) control. Alternatively, the number of nuclei may be in terms of fold difference; for example, the number of nuclei in the test sample may be at least about 2 fold, at least about 3 fold, at least about 4 fold, at least about 5 fold, at least about 8 fold, or at least about 10 fold times higher of the normal (non-prostate cancer) control. In contrast, a similar number of nuclei in a test prostate sample relative to a normal control sample or a control prostate cancer sample may mean that the number of nuclei in the test sample differs by no more than 5%, no more than 2%, or more than 1%, such as 0.5-5% of the normal or prostate cancer control.

An decrease in the size of lumen relative to a normal sample may mean, for example, that the average lumen size the test sample is at least at least 15%, at least 20%, at least 25%, at least 30%, at least 50%, at least 75%, at least 80%, at least 90%, or at least 98% lower of the normal control. Alternatively, the average lumen size may be in terms of fold difference; for example, the average lumen size in the test sample may be at least about 2 fold, at least about 3 fold, at least about 4 fold, at least about 5 fold, at least about 8 fold, or at least about 10 fold times lower of the normal control. In contrast, similarly size lumen in a test prostate sample relative to a normal sample or a prostate cancer sample may mean that the average lumen size in the test sample differs by no more than 5%, no more than 2%, or more than 1%, such as 0.5-5% of the normal or prostate cancer control. In some examples, lumen size is characterized by the radius (major and minor axis) of the elliptical lumen shape. Such radius may also be the average of the two major and minor radii.

### Exemplary Methods

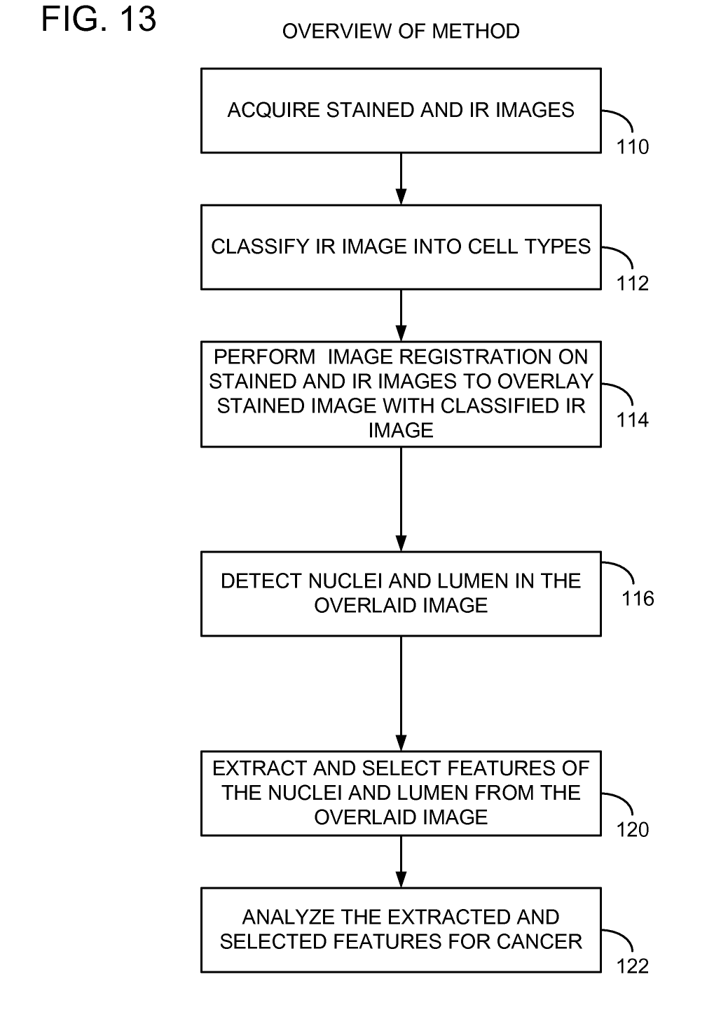

FIGS. 13-16 illustrate a method for detecting or diagnosing prostate cancer. Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

Any of the disclosed methods can be implemented as computer-executable instructions stored on one or more computer-readable media (e.g., non-transitory computer-readable media, such as one or more optical media discs, volatile memory components (such as DRAM or SRAM), or nonvolatile memory components (such as hard drives)) and executed on a computer (e.g., any commercially available computer, including smart phones or other mobile devices that include computing hardware). Any of the computer-executable instructions for implementing the disclosed techniques as well as any data created and used during implementation of the disclosed embodiments can be stored on one or more computer-readable media (e.g., non-transitory computer-readable media). The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, JavaScript, Adobe Flash, or any other suitable programming language. Likewise, the disclosed technology is not limited to any particular computer or type of hardware. Certain details of suitable computers and hardware are well known and need not be set forth in detail in this disclosure.

The disclosed methods, apparatus, and systems should not be construed as limiting in any way. Instead, the present disclosure is directed toward all novel and nonobvious features and aspects of the various disclosed embodiments, alone and in various combinations and subcombinations with one another. The disclosed methods, apparatus, and systems are not limited to any specific aspect or feature or combination thereof, nor do the disclosed embodiments require that any one or more specific advantages be present or problems be solved.

Turning to FIG. 13, in process block 110, stained and IR images are acquired. For example, the stained image can be acquired using an H&E stained image while the IR image can be obtained using FT-IR. In process block 112, each of the images is classified. The classification process is used to classify the data into cell types. In process block 114, an image registration is performed on the stained and IR images to overlay the stained image with the IR image. Image registration is the process of transforming the different sets of data into one coordinate system. Registration is desirable in order to be able to compare or integrate the data obtained from different measurements. In process block 116, nuclei and lumen in the overlaid image are detected. In process block 120, features of the nuclei and lumen are extracted and selected. In process block 122, the extracted and selected features are analyzed to determine if they are cancerous.

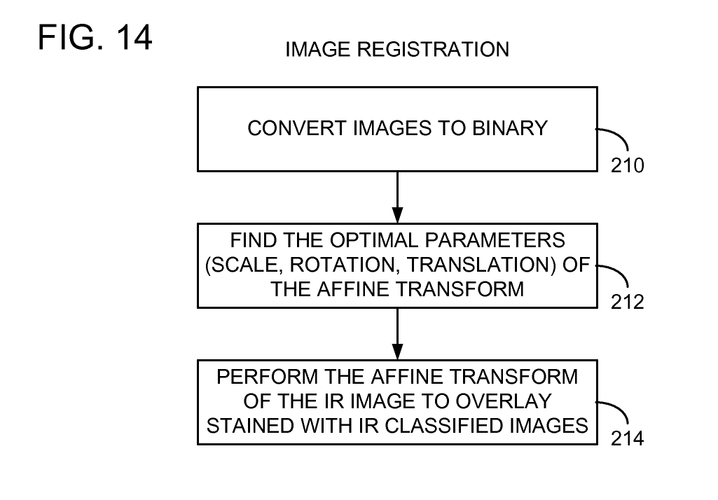

FIG. 14 is a flowchart of a method showing an example image registration of process block 114. In process block 210, the images are converted to binary. In process block 212, the optimal parameters (scale, rotation, and translation) are determined of an affine transformation on the IR image. In process block 214, the affine transformation is performed to overlay stained with IR classified images.

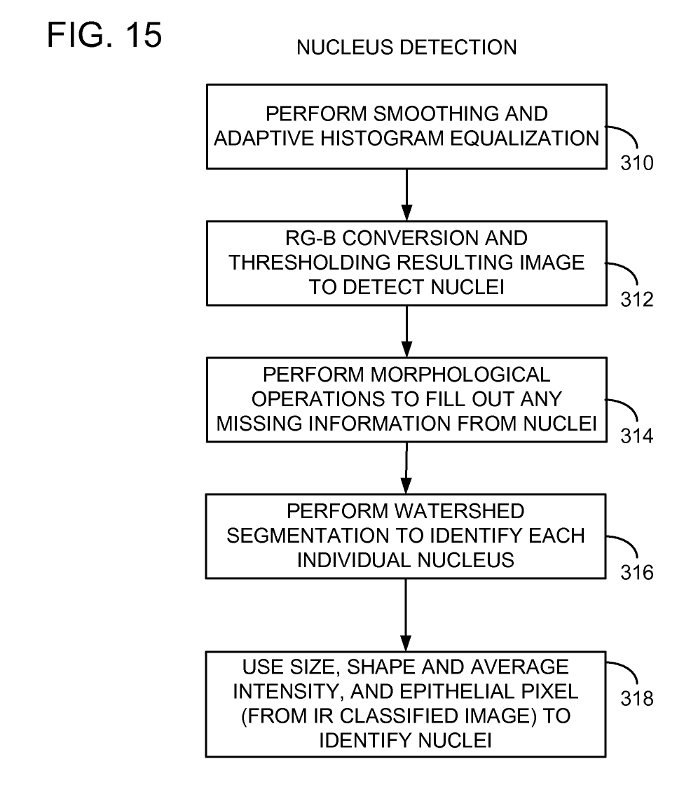

FIG. 15 is a flowchart of a method showing an example of nuclei detection of process block 118. In process block 310, a smoothing and an adaptive histogram equalization are performed. In process block 312, RG−B conversion and thresholding of the resulting image are performed to detect nuclei. In process block 314, morphological operations are performed in order to fill out any missing information from the nuclei. In process block 316, watershed segmentation is performed to identify each individual nucleus. In process block 318, the size, shape and average intensity, and epithemlial pixel are used to identify the nuclei.

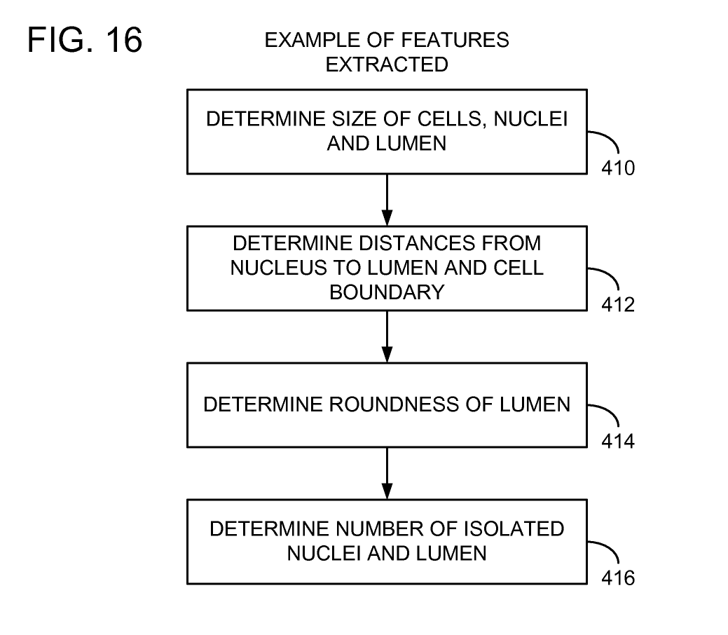

FIG. 16 is a flowchart of a method for extracting features from nuclei and lumen. There are a variety of extraction methods that can be used. FIG. 4 shows some examples of features that can be extracted. In process block 410, the size of cells, nuclei and lumen can be determined. In process block 412, distances from the nucleus to the lumen and cell boundaries can be determined. In process block 414, the roundness of the lumen can be determined. In process block 416, the number of isolated nuclei and lumen can be determined. The feature categories can generally be described in 5 groups—size, number, distance, shape and distribution. The following list is another possible alternative of some of the features.
1) size of epithelial cells, nuclei, and lumen
2) number of nuclei (total, isolated, and far), lumen
3) distance from nucleus to lumen and cell boundary
4) lumen shape: distortion, roundness, minimum bounding circle ratio, convex ratio, symmetric index of lumen boundary and area
5) spatial distribution: entropy of spatial distribution of nuclei and spatial association of lumen and cytoplasm

### EXAMPLE 1

### Methods

Chemical and morphologic data were recorded from an unstained tissue microarray (TMA) using Fourier transform infrared (FT-IR) spectroscopic imaging. Using pattern recognition, epithelial cells were identified without user input. The spatial information was fused with the corresponding stained images commonly used in clinical practice. Extracted morphological features, optimized by two-stage feature selection method using a minimum-redundancy-maximal-relevance (mRMR) criterion and sequential floating forward selection (SFFS), were applied to classify samples as cancer or non-cancer.

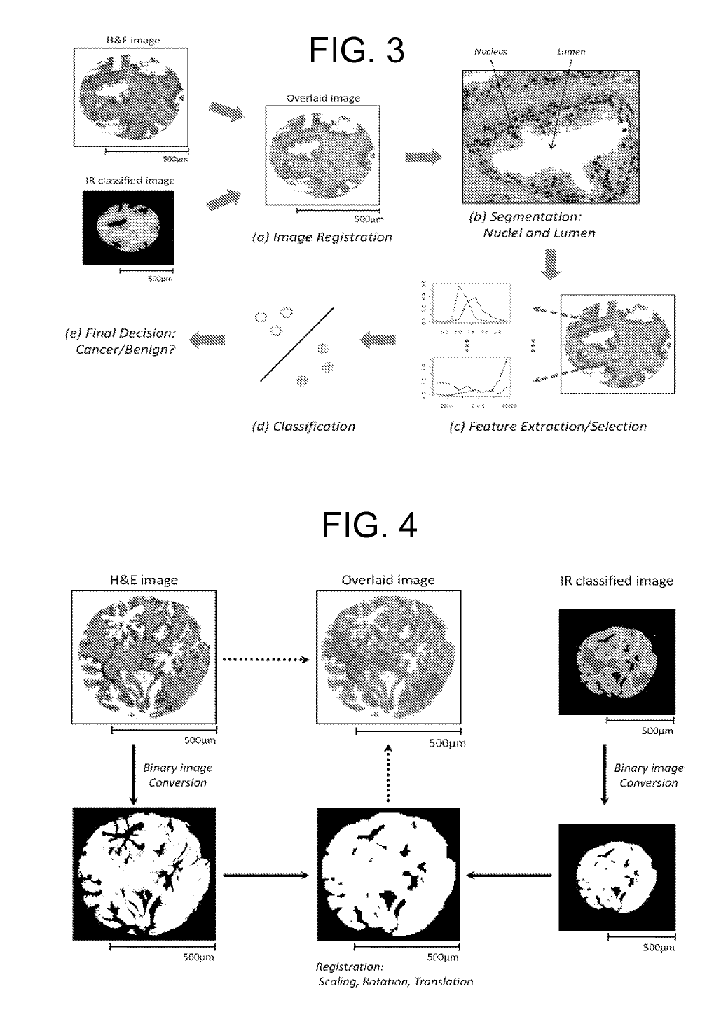

Provided below is a description of the method. One aspect of the method is the use of FT-IR imaging data on a serial section that is H&E-stained to enhance the segmentation of nuclei and lumens. The first two components (§1-2) are geared to this functionality, while the next three components (§3-5) exploit the segmented features obtained from image data to classify the tissue sample (FIG. 3).

### 1. Image Registration

Given two images, the image registration problem can be defined as finding the optimal spatial and intensity transformation [28] of one image to the other. Here, two images are H&E stained and “IR classified” images which were acquired from adjacent tissue samples. The IR classified image represents the FT-IR imaging data, processed as indicated in FIG. 2, to classify each pixel as a particular cell type. Although the two samples were physically in the same intact tissue and are structurally similar, the two images have different properties (total image and pixel sizes, contrast mechanisms and data values). Hence, features to spatially register the images are not trivial.

The H&E image provides detailed morphological information that could ordinarily be used for registration, but the IR image lacks such information. On the other hand, the IR image specifies the exact areas corresponding to each cell type, but the difficulty in precisely extracting such regions from the H&E image hinders using cell-type information for registration. The features used were macroscopic sample shape and empty space (lumens) inside the samples. To utilize these two features and to avoid problems due to differences in the two imaging techniques, both images are first converted into binary images. Due to the binarization, the intensity transformation is not necessary. As a spatial transformation, an affine transformation (f) was used [28] where a coordinate (x₁, y₁) is transformed to the (x₂, y₂) coordinate after translations (tₓ, t_{y}), rotation by θ, and scaling by factor s.

$$[ \begin{matrix}{x}_{2} \\ {y}_{2}\end{matrix} ] = [ \begin{matrix}{t}_{x} \\ {t}_{y}\end{matrix} ] + s [ \begin{matrix}cos θ & - sin θ \\ sin θ & cos θ\end{matrix} ] [ \begin{matrix}{x}_{1} \\ {y}_{1}\end{matrix} ]$$

Accordingly, the optimal parameters of the affine transformation that minimizes the absolute intensity difference between two images (I_{reference} and I_{target}) are identified. In other words, image registration amounts to finding the optimal parameter values

$$( {t}_{x}^{*} , {t}_{y}^{*} , {θ}^{*} , {s}^{*} ) = {arg min}_{{t}_{x} , {t}_{y} , θ , s} \vert {I}_{reference} - f ( {I}_{target} ; {t}_{x} , {t}_{y} , θ , s ) \vert .$$

The downhill simplex method [29] is applied to solve the above equation. An example of this registration process is shown in FIG. 4.

More specific details on the image registration methods are provided below.

In order to map the cell type information from IR classified images on H&E images, the image registration, the process of finding the optimal spatial and intensity transformation of one image (H&E image; I_{reference}) to the other (IR classified image; I_{target}) was needed. Two tissue samples were physically in the same intact tissue and are structurally similar. Macroscopic sample shape and empty space (lumens) inside the samples are well matched between two images. However, due to differences in two imaging techniques, two images have different properties (total image and pixel size, contrast mechanisms and data values) and specify different information; H&E images provide detailed morphological information whereas IR classified images contain cell type information. Intensity values of each pixel in two images are also greatly different. Each pixel in H&E images has 3 channels (Red, Green, and Blue) ranging from 0 to 255, and, in IR classified images, a label indicating its cell type is assigned to each pixel.

Intensity differences were eliminated prior to registration by using an affine transformation as a spatial transformation, and to estimate the transformation from the entire image. To eliminate the intensity difference between H&E image and IR classified image, both images are converted into binary images, i.e., pixels representing a tissue are assigned “1” and other pixels including lumens are set to “0”. For IR classified image, pixels labeled with cell types is the ones representing a tissue. Accordingly, assigning “1” to those pixels and “0” to others completes the binarization. For H&E image, we use a proper threshold value (>200) for the intensity of Red (R), Green (G), and Blue (B) channels since both lumens and background regions are white. Then, inverting the thresholded image gives the binary image of H&E image. As a result of the binarization, the intensity transformation is unnecessary. Binarization does not alter the geometrical characteristics (macroscopic shape and lumens) of the two images. The affine transformation (f) transforms a coordinate (x₁, y₁) to the (x₂, y₂) coordinate after translations (tₓ, t_{y}), rotation by θ, and scaling by factor s.

$$[ \begin{matrix}{x}_{2} \\ {y}_{2}\end{matrix} ] = [ \begin{matrix}{t}_{x} \\ {t}_{y}\end{matrix} ] + s [ \begin{matrix}cos θ & - sin θ \\ sin θ & cos θ\end{matrix} ] [ \begin{matrix}{x}_{1} \\ {y}_{1}\end{matrix} ]$$

Since two adjacent tissue samples are structurally similar, it is assumed that two images do not suffer from large deformation, and the affine transformation is sufficient to model the geometrical change between two images. Difficulty in extracting features, ascribed to different properties and information provided by two images, leads us to use entire image for estimating the transformation parameters. The absolute intensity difference between two images is defined as error metric (or similarity measure). The absolute intensity difference between two images is, in fact, corresponding to the total number of pixels where two images have different labels owing to binarization. The better registration, the smaller number of those pixels it results in. Thus, the optimal registration can be obtained by minimizing the absolute intensity difference between two images. In other words, the image registration amounts to finding the optimal parameter values of the affine transformation

$$( {t}_{x}^{*} , {t}_{y}^{*} , {θ}^{*} , {s}^{*} ) = {arg min}_{{t}_{x} , {t}_{y} , θ , s} \vert {I}_{reference} - f ( {I}_{target} ; {t}_{x} , {t}_{y} , θ , s ) \vert$$

To reduce the search space, the center of two images is aligned and scaled up I_{target} by estimating the radius of both samples. Afterwards, random samples of the parameter values are drawn, the coordinate of I_{target} transformed, and the absolute intensity difference to obtain the initial solution compute. Then, the downhill simplex method [29] is applied to attain the final solution.

In order to quantitatively validate the accuracy of the method, experiments were conduced using one IR classified image and simulated images. The simulated images are generated by transforming the given IR classified image with different parameter values: 1) scaling factor s in the range [0.5, 1.5], 2) rotation angle θ in the range

$$[ - \frac{π}{2} , \frac{π}{2} ] , 3 )$$

translation (tₓ, t_{y}) in the range [−50, 50]. For each of the three cases, 100 simulated images are generated, and another 100 images are also generated by varying all parameters simultaneously. After applying the registration method to register the IR classified image with the simulated images, the true parameters were compared with the recovered parameters by computing registration error (the absolute difference between parameters). As shown in Table 1, the registration method well recovers the true parameters. Therefore, the registration method can successfully register the H&E image with the IR classified image in the absence of large deformation.

TABLE 1
Registration results with simulated images.*

| Varied Parameters | Registration Error (s, θ, tₓ, t_{y}) |
|---|---|
| s | (0.0109, 0.4735, 0.7705, 0.6874) |
| θ | (0.0042, 0.4941, 0.0991, 0.0900) |
| tₓ, t_{y} | (0.0028, 0.0662, 0.5068, 0.5734) |
| s, θ, tₓ, t_{y} | (0.0097, 3.4416, 0.9626, 0.7353) |

*For each case, the average registration error in the recovered parameters is computed over 100 simulated images. Scaling s, rotation angle θ, and translation (tₓ, t_{y}) errors are given relative to the original image scale, in degrees, and in pixels, respectively.

### 2. Identification of Epithelial Cells and Their Morphologic Features

While a number of factors are known to be transformed in cancerous tissues, epithelial morphology is utilized as the clinical gold standard. Hence, the focus was on cellular and nuclear morphology of epithelial nuclei and lumens. These structures are different in normal and cancerous tissues, but are not widely used in automated analysis for a few reasons. First, simple detection of epithelium from H&E images is difficult. Second, detection of epithelial nuclei may be confounded by a stromal response that is not uniform for all grades and types of cancers. The focus was to address these two challenges that hinder automatically parsing morphologic features such as the size and number of epithelial nuclei and lumens, distance from nuclei to lumens, geometry of the nuclei and lumens, and others (§3). In order to use these properties, the first step is to detect nuclei and lumens correctly using a robust strategy.

### 2.1. Lumen Detection

In H&E stained images, lumens are recognized to be empty white spaces surrounded by epithelial cells. In normal tissues, lumens are larger in diameter and can have a variety of shapes. In cancerous tissues, lumens are progressively smaller with increasing grade and generally have less distorted elliptical or circular shapes. Thus, to detect lumens, empty areas located next to the areas rich in epithelium were located. White spots inside the sample can be found from the H&E image, and the pixels corresponding to epithelial cells can be mapped on the H&E image from the IR classified image through image registration. While lumens are ideally completely surrounded by epithelial cells (called complete lumens), some samples have lumens (called incomplete lumens) that violate this criterion because only a part of lumen is present in the sample. To identify these incomplete lumens, heuristic criteria based on the size, shape, presence of epithelial cells and background around the areas, and distance from the center of the tissue was used.

Additional information on how lumens were detected is provided below.

Complete lumen detection starts from identifying white spots inside the samples from the H&E image by using a proper threshold value (>200) for the intensity of Red (R), Green (G), and Blue (B) channels. The white spots may include many artifacts which are, in our observations, relatively small and/or have narrowly elongated needle-like shape. Owing to IR overlay, pixels corresponding to epithelial cells from the IR classified image can be mapped on the H&E image, and it allows identification of artifactual lumens, which are not associated epithelial cells. By definition, lumens are surrounded by epithelial cells. Each white spot is examined to determine whether more than 30% of its perimeter is next to or within the areas where epithelial pixels are present. If the condition is not satisfied, the spot is considered to be an artifact. To further prune the white areas that passed the condition, a simple rule, restricting the size and shape, is invoked: If the size of any white area is smaller than 10 pixels or the major and minor axis ratio (r_{major/minor}) is greater than 3 when its size is smaller than 100 pixels, the white area also is considered to be an artifact. Lumens are progressively smaller and lesser distorted elliptical or circular with increasing grade; that is, r_{major/minor} is getting closer to 1, and larger r_{major/minor} is indicative of artifact. r_{major/minor} is computed by using the major and minor axes of an ellipse fitted to each white area.

Since each tissue sample is a small portion of an entire tissue, the tissue sample often includes lumens that do not form a complete geometrical shape (incomplete lumens). Their perimeter is adjacent to either epithelial cells or to background. The fraction of the lumen's perimeter that is adjacent to background is relatively small. However, without examining the original tissue that the samples were taken from, it is impossible to infer the original size and shape of such incomplete lumens. To handle this problem, an entire tissue sample is modeled as a circle, and the white spots between the tissue sample and the circle are the candidate incomplete lumens. The same threshold value (>200) for the complete lumen detection is used to identify candidate white areas which may include artifactual lumens. The artifactual incomplete lumens are relatively small and/or in crescent shapes along the edge of tissues. Crescent-like artifacts result from the gaps between the tissue sample and the circle fitted to the sample, and their average distance from the center of the sample is close to the radius of the sample. Based on these observations, similar to the artifactual complete lumens, the white areas are restricted by the following considerations: the fraction of their perimeter bordering epithelial cells must be >0.65 and that bordering background must be <0.4, their size must be greater than 100 pixels, the shape must have r_{major/minor}<3, and the average distance of their perimeter to the center of the tissue must be less than 90% the radius of the tissue core.

### 2.2. Nucleus Detection—Single Epithelial Cells

Epithelial nucleus detection by automated analysis is more difficult than lumen detection due to variability in staining and experimental conditions under which the entire set of H&E images were acquired. Differences between normal and cancerous tissues, and among different grades of cancerous tissues, also hamper facile detection. To handle such variations and make the contrast of the images consistent, smoothing [30] and adaptive histogram equalization [31] were used prior to nuclei identification.

Nuclei are relatively dark and can be modeled as small elliptical areas in the stained images. This geometrical model is often confounded as multiple nuclei can be so close as to appear like one large, arbitrary-shaped nucleus. Also, small folds or edge staining around lumens can make the darker shaded regions difficult to analyze. Here, the information provided by the IR classified image was used limit the analysis to epithelial cells, and a thresholding heuristic on a color space-transformed image used to identify nuclei with high accuracy.

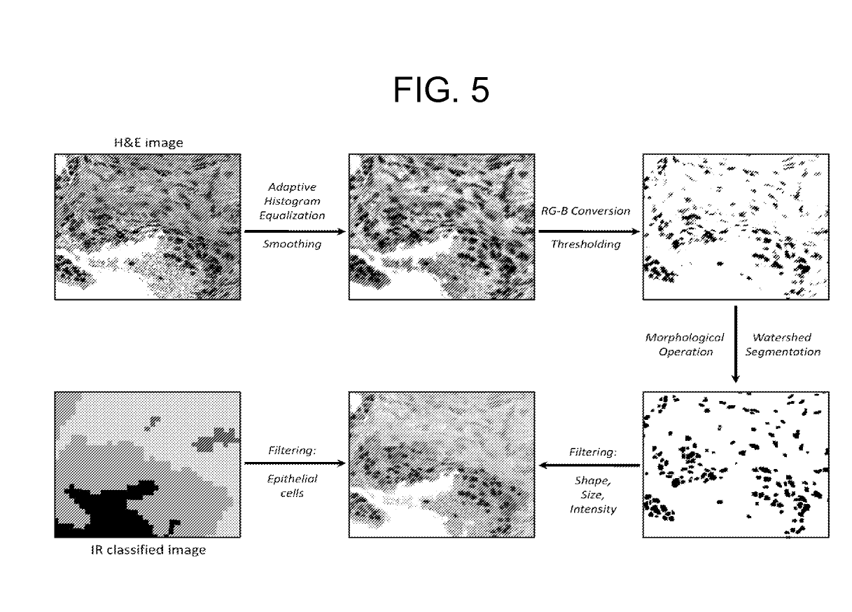

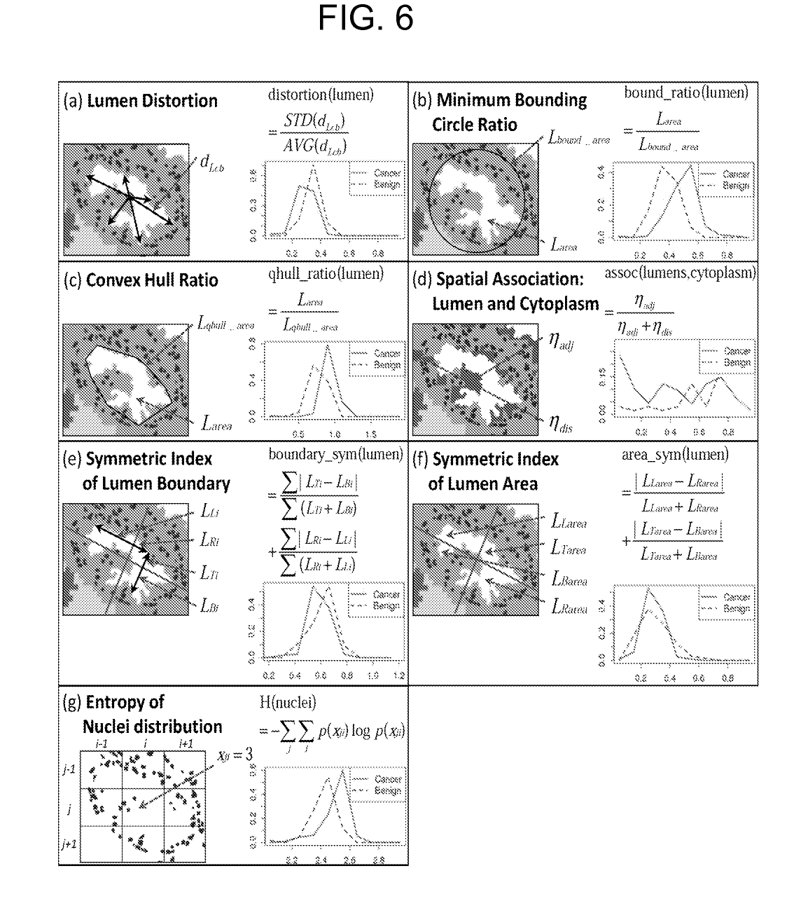

Epithelial pixels that are identified on the H&E images using the IR overlay provide pixels of dominated by one of two colors: blue or pink, which arise from the nuclear and cytoplasmic component respectively. For nuclei restricted to epithelial cells in this manner, a set of general observations were made that led us to convert the stained image to a new color space “RG−B” (|R+G−B|). (R, G, and B represent the intensity of Red, Green, and Blue channels, respectively.) This transformation, followed by suitable thresholding, was able to successfully characterize the areas where nuclei are present. The threshold values are adaptively determined for Red and Green channels due to the variations in the color intensity. Finally, filling holes and gaps within nuclei by a morphological closing operation [32], the segmentation of each nucleus is accomplished by using a watershed algorithm [32] followed by elimination of false detections. The size, shape, and average intensity are considered to identify and remove artifactual nuclei. FIG. 5 details the nucleus detection procedure.

Additional information on how nuclei are detected is provided below.

Nuclei are modeled as relatively dark and small elliptical areas in the stained images. It was observed that both blue and red channel intensity of pixels corresponding to epithelial cells, nuclear components in particular, do not suffer from the variability as much as green channel intensity. The green channel intensity varies a lot from image to image; for example, its histogram is highly skewed in cancerous tissues. This may increase a false discovery of nuclei in cancerous cells.

To overcome the problem, the segmentation was made consistent and robust, and to obtain better contrast, the stained image was smoothed [30] and adaptive histogram equalization [31] applied to green channel. Adaptive histogram equalization is an image enhancement technique which redistributes each pixel value proportional to the intensities of its surrounding pixels. Because applying adaptive histogram equalization to all the three channels could bring dramatic alterations and biases in color spaces, w only green channel possessing the highest deviation was applied. As mentioned above, epithelial pixels mapped on the stained images using the IR overlay can provide nuclear and cytoplasmic pixels. Examining nuclei restricted to epithelial cells, a set of general observations may be noted: 1) Red, Green, and Blue channel intensities are lower in nuclear pixels and higher in cytoplasmic pixels. 2) Green channel intensity is lower than other channels in both cytoplasmic and nuclear pixels. 3) In stromal cells, which are not considered here, Red channel intensity is usually higher than other channels. 4) A difference between Red and Blue channel intensities is small both in cytoplasmic and nuclear pixels.

Based on these observations, it was observed that converting the stained image to a new image where each pixel has an intensity value |R+G−B| could well characterize the areas where epithelial nuclei are present. In RG−B space, nuclear pixels mostly have lower values than cytoplasmic pixels and pixels belonging to other cell types such as stroma. During the color space conversion, a few intensity constraints are imposed on Green and Red channels. For both Green and Red channels, the threshold values (Th_{Red} and Th_{Green}) are computed by

$$AVG ( P ) - \frac{2}{3} STD ( P ) ,$$

respectively. P represents a set of pixels where Red channel intensity is less than either of two other channels (avoid to include stromal pixels) and AVG(•) and STD(•) represent the average and standard deviation. Adaptively computed threshold values may help to manage variations in the stained images. Green channel intensity is required to be less than Th_{Green} and Red channel intensity is required to be less than Th_{Red} or other two channel intensities. Restriction imposed on Red channel is to eliminate pixels corresponding to stromal cells, just in case that the IR overlay fails.

After the color space conversion, a morphological closing operator [32] is applied to the image to fill small holes and gaps within nuclei, and the segmentation of each individual nucleus is accomplished by using watershed algorithm [32]. To alleviate possible over-segmentation of the nuclei (Roerdink J B T M, Meijster A, Fundam Inf 2000, 41:187-228), we expand each segmented nucleus area N_{seg} by including all neighboring pixels whose intensities falling within AVG(N_{seg})±STD(N_{seg}). Although properly determined, the segmentation may include many false predictions. To refine the segmentation, each individual nucleus is constrained by its shape and size: r_{major/minor}<4 and size of a nucleus >5 and <2× median size of all nuclei. In addition, the average intensity of a nucleus is restricted to be less than Th_{Green}. The nuclei that satisfy all the conditions and are located within the epithelial cells are reported as epithelial nuclei.

### 2.3. Epithelium Detection

Epithelium was detected as follows. In epithelial cells, two types of pixels can be observed—nuclear and cytoplasmic pixels. The strategy to detect epithelial pixels from the H&E stained images was to identify cytoplasmic pixels since nuclei can be detected by the above method. The set of observations made for epithelial cells above is useful for cytoplasmic pixel detection. In addition to the observations, it is noted that the ratio of blue channel intensity to sum of all channel intensity is quite high for cytoplasmic pixels. Hence, the value of each pixel was computed as follows:

$$B ( 1 + \frac{B}{R + G + B} ) - G$$

It emphasizes the pixels that have both higher intensity and relatively higher ratio of blue channel and have lower green channel intensity, and such pixels are cytoplasmic pixels in general. The segmentation of cytoplasmic areas is performed by finding a threshold value iteratively (Picture Thresholding Using an Iterative Selection Method. Systems, Man and Cybernetics, IEEE Transactions on 1978, 8:630-632). At iteration i, a threshold value is updated as

$${T}_{i} = \frac{1}{2} ( {μ}_{i}^{1} + {μ}_{i}^{2} )$$

where μᵢ¹ and μᵢ² denote the average values of two sets of pixels grouped by the threshold value Tᵢ₋₁. One set contains the pixels whose values are greater than Tᵢ₋₁ (cytoplasmic areas) and the pixels in the other set has the values less than Tᵢ₋₁. The thresholding method may not capture all the cytoplasmic areas. Each cytoplasmic area C_{seg} is grown by finding the adjacent pixels within

$$AVG ( {C}_{seg} ) \pm \frac{1}{2} STD ( {C}_{seg} ) .$$

Small holes are identified and filled inside of each segment to include pixels representing epithelial nuclei. The segmented image often contains many salt and pepper type noise. To remove them, median filter (Huang et al., A fast two-dimensional median filtering algorithm. Acoustics, Speech and Signal Processing, IEEE Transactions on 1979, 27:13-18) is applied to the segmented image. As did in nucleus detection, adaptive histogram equalization is applied to green channel to deal with variability in the stained images prior to epithelium detection.

### 3. Feature Extraction

The characteristics of nuclei and lumens change in cancerous tissues. In a normal tissue, epithelial cells are located mostly in thin layers around lumens. In cancerous tissue, these cells generally grow to fill lumens, resulting in a decrease in the size of lumens, with the shape of lumens becoming more elliptical or circular. The epithelial association with a lumen becomes inconsistent and epithelial foci may adjoin lumens or may also exist without an apparent lumen. Epithelial cells invading the extra-cellular matrix also result in a deviation from the well-formed lumen structure; this is well-recognized as a hallmark of cancer. Due to filling lumen space and invasion into the extra-cellular space, the number density of epithelial cells increases in tissue. The size of individual epithelial cells and their nuclei also tend to increase as malignancy of a tumor increases. Due to these recognized morphological differences between normal and cancerous tissues, epithelial nuclei and lumens were used as the basis of the several quantitative features that the disclosed classification system works with. (See examples of such features in FIG. 6.) These observations are qualitative in actual clinical practice and have not been previously quantified.

### 3.1. Epithelial Cell-Related Features

Epithelial cell type classification from IR data was used to measure epithelium-related features. However, individual epithelial cells in the tissue are not easily delineated. Therefore, in addition to features directly describing epithelial cells, properties of epithelial nuclei, which are available from the segmentation described in §2, were quantified. The quantities measured in defining features are: (1) size of epithelial cells, (2) size of epithelial nuclei, (3) number of nuclei in the sample, (4) distance from a nucleus to the closest lumen, (5) distance from a nucleus to the epithelial cell boundary, (6) number of “isolated” nuclei (nuclei that have no neighboring nucleus within a certain distance), (7) number of nuclei located “far” from lumens, and (8) entropy of spatial distribution of nuclei (FIG. 6G).

Provided below are the specifics of these measures and their calculation. The list of names and meanings of epithelium related features are:
1) Size of Epithelial cells: Size of epithelial cells.
2) Size of a Nucleus: Size of a nucleus.
3) Number of Nuclei: Number of nuclei.
4) Distance to Lumen: Distance from the center of a nucleus to the boundary of the closest lumen.
5) Distance to Epithelial Cell Boundary: Epithelial cell boundaries are estimated by drawing a Voronoi diagram of the segmented epithelial regions (obtained from IR image) with the segmented nuclei serving as the Voronoi sites. The cell corresponding to each nucleus, also called the Voronoi cell, comprises all points that are closer to that nucleus than to any other nuclei. The Voronoi cell of a nucleus is considered as the epithelial cell to which the nucleus belongs, and the distance to the epithelial cell boundary is the distance from the center of the nucleus to the boundary of its Voronoi cell.
6) Number of Isolated Nuclei [19]: Number of nuclei without having a neighboring nucleus within a distance D_{Iso} (20 um) from the center of each nucleus.
7) Fraction of Distant Nuclei: Fraction of nuclei away from lumens. If the distance from a nucleus to the boundary of the closest lumen is greater than D_{Dis} (30 um), the nucleus is called a distant nucleus.
8) Entropy of Nuclei Spatial Distribution: To measure the entropy of nuclei spatial distribution, an entire tissue is divided into N×N equal-sized partitions and the number of nuclei in each partition is counted. The entropy is computed as follows:

$$H ( Nuclei ) = - {\sum }_{i = 1}^{n} {\sum }_{j = 1}^{n} p ( {x}_{ij} ) log p ( {x}_{ij} )$$

p( ) denotes the probability mass function of the number of nuclei in a partition. xᵢⱼ denotes the number of nuclei in (i,j)th partition.
3.2. Lumen-Related features

Features describing glands have been shown to be effective in prostate cancer classification [18] [21]. Here, lumens were characterized by focusing on the differences in the shape of the lumens. The quantities measured in defining these features are: (1) size of a lumen, (2) number of lumens, (3) lumen “roundness” [21], defined as

$$\frac{{L}_{peri}}{2 {L}_{area}} r$$

where Lₚₑᵣᵢ is the perimeter of the lumen, Lₐᵣₑₐ is the size of the lumen, and r is the radius of a circle of size Lₐᵣₑₐ, (4) lumen “distortion” (FIG. 6A), computed as

$$\frac{STD ( {d}_{{L}_{cb}} )}{AVG ( {d}_{{L}_{cb}} )}$$

where d_{L}_{_{cb}} is the distance from the center of a lumen to the boundary of the lumen and AVG(•) and STD(•) represent the average and standard deviation, (5) lumen “minimum bounding circle ratio” (FIG. 6B), defined as the ratio of the size of a minimum bounding circle of a lumen to the size of the lumen, (6) lumen “convex hull ratio” (FIG. 6C), which is the ratio of the size of a convex hull of a lumen to the size of the lumen, (7) symmetric index of lumen boundary (FIG. 6E), (8) symmetric index of lumen area (FIG. 6F), and (9) spatial association of lumens and cytoplasm-rich regions (FIG. 6D). Features (3)-(8) are various ways to summarize lumen shapes, while feature (9) is motivated by the loss of functional polarization of epithelial cells in cancerous tissues.

Additional information on how the lumen-related features are provided below. The names and meanings of lumen related features are:
1) Size of a Lumen: Number of pixels in a lumen.
2) Number of Lumens: Number of lumens in a tissue.
3) Lumen Roundness [16]: Roundness of a lumen is defined as

$$\frac{{L}_{peri}}{2 {L}_{area}} r$$

where Lₚₑᵣᵢ is the perimeter of the lumen, Lₐᵣₑₐ is the size of the lumen, and r is the radius of a circle with the size of Lₐᵣₑₐ.
4) Lumen Distortion: Distortion of a lumen is computed as

$$\frac{STD ( {d}_{{L}_{cb}} )}{AVG ( {d}_{{L}_{cb}} )}$$

where d_{Lcb} is the distance from the center of a lumen to the boundary of the lumen.
5) Lumen Minimum Bounding Circle Ratio: Ratio of the size of a minimum bounding circle of a lumen to the size of the lumen.
6) Lumen Convex Hull Ratio: Ratio of the size of a convex hull of a lumen to the size of the lumen.
7) Symmetric Index of Lumen Boundary: Sum of Vertical and Horizontal Symmetry. Vertical and Horizontal Symmetry are defined as

$$\frac{\sum \vert {L}_{Ti} - {L}_{Bi} \vert}{\sum ( {L}_{Ti} + {L}_{Bi} )} and \frac{\sum \vert {L}_{Ri} - {L}_{Li} \vert}{\sum ( {L}_{Ri} + {L}_{Li} )} ,$$

respectively. L_{Ti} and L_{Bi} are vertical distances from a vertical axis to the boundary of the lumen, L_{Li} and L_{Ri} are horizontal distances from a horizontal axis to the boundary of the lumen. The vertical axis runs along the longest diameter, and the horizontal axis runs perpendicularly to the horizontal axis passing the center of the lumen.
8) Symmetric Index of Lumen Area: Sum of Left-Right Area Symmetry and Top-Bottom Area Symmetry. Left-Right and Top-Bottom Area symmetry are computed as

$$\frac{\vert {L}_{Larea} - {L}_{Rarea} \vert}{{L}_{Larea} + {L}_{Rarea}} and \frac{\vert {L}_{Tarea} - {L}_{Barea} \vert}{{L}_{Tarea} + {L}_{Barea}} ,$$

respectively. L_{Larea}, L_{Rarea}, L_{Tarea}, and L_{Barea} are the size of left, right, top, and bottom quadrants, respectively. These quadrants are obtained by dividing the lumen through imaginary vertical or horizontal axes. The vertical and horizontal axes are defined as in 7).
9) Spatial Association of Lumens and cytoplasm-rich regions: Spatial association of lumens and cytoplasm-rich regions is computed as

$$\frac{{η}_{adj}}{{η}_{adj} + {η}_{dis}}$$

where η_{dis} is a set of cytoplasm-rich pixels distant to lumens and η_{adj} is a set of cytoplasm-rich pixels adjacent to lumens. The process of obtaining cytoplasm-rich pixels is provided in [11]. To obtain adjacent cytoplasm-rich pixels, the pixels were first searched around the boundary of lumens, and if a cytoplasm-rich pixel found, then neighboring cytoplasm-rich pixels are searched. These are repeated until no more cytoplasm-rich pixels are found.

### 3.3. Global & Local Tissue Features

Described above are the individual measures of epithelium and lumen related quantities that form the basis of the features used by the disclosed classification system.

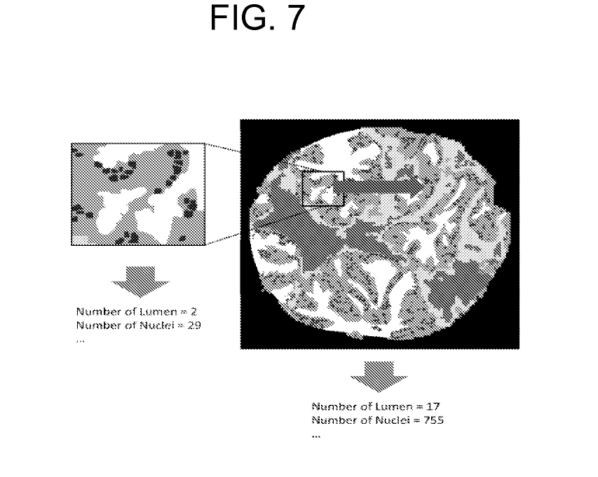

Normally, these features are summary measures over the entire tissue sample or desired classification area. Hence, average (AVG) or standard deviation (STD), and in some cases the sum total (TOT) of these quantities, are employed for further analysis. These features are called “global” features since they are calculated from the entire sample. However, in some cases global features may be misleading, especially where only a part of the tissue sample is indicative of cancer. Therefore, in addition to global features, we “local” features are defined by sliding a rectangular window of a fixed size (typically 100×100 pixels) throughout a tissue sample, computing the average or sum total of the feature in each window, and computing the standard deviation and/or extrema over the values for all windows (FIG. 7). In all, 67 features (29 global and 38 local features) are defined capturing various aspects of tissue morphology. These 67 features, or a subset thereof, can be used to generate the final classified.

### 4. Feature Selection

Feature selection is the step where the classifier examines all available features (in some examples the 67 features discussed herein) with respect to the training samples, and selects a subset to use on test data. This selection is generally based on the criterion of high accuracy on training data, but also strives to ensure generalizability beyond the training data. A two-stage feature selection approach was used. In the first stage, a set of candidate features (C_{candidate}) is generated using the minimum-redundancy-maximal-relevance (mRMR) criterion [33]. In each iteration, given a feature set chosen thus far, mRMR chooses the single additional feature that is least redundant with the chosen features, while being highly correlated with the class label.

mRMR [33] is a feature selection method based on mutual information. It attempts not only to maximize the relevance between selected features and a class label, but also to minimize the redundancy between selected features. Since a set of best features does not result in the best feature set, eliminating redundant features is important to provide a good subset of features. Both relevance and redundancy are characterized in terms of mutual information as follows:

maximal relevance: max D(S, c),

$$D = \frac{1}{\vert S \vert} {\sum }_{{x}_{i} ∈ S} I ( {x}_{i} ; c )$$

minimal redundancy: max R(S),

$$R = \frac{1}{{\vert S \vert}^{2}} {\sum }_{{x}_{i} , {x}_{j} ∈ S} I ( {x}_{i} ; {x}_{j} )$$

where I(x,y) is the mutual information of two variables x and y, S is the feature set, and c is the class label. To achieve the goal of optimizing above two conditions simultaneously, the simple mRMR criterion, max (D−R), is invoked. It starts from a feature with the highest maximal relevance, and a new feature is selected and added to the current feature set if it satisfies the mRMR critetion among the rest of features. Thus, it generates, in fact, the order of the features according to the mRMR criterion.

C_{candidate} is a set of features that is expected to be close to the optimal feature set for a dataset and a classifier under consideration. It is constructed as follows. Given a feature set F=(f₁, . . . , f_{M}) ordered by mRMR, AUC of the set of i top-ranked features is computed for varying values of i. The value of i is set to ≦30. The feature subset with the best AUC is chosen as the C_{candidate}.

In the second stage, feature selection continues with C_{candidate} as the starting point, using the sequential floating forward selection (SFFS) method [34]. This method sequentially adds new features followed by conditional deletion(s) of already selected features. Starting with the C_{candidate}, SFFS searches for a feature x∉C_{candidate} that maximizes the AUC among all feature sets C_{candidate}∪{x}, and adds it to C_{candidate}. Then, it finds a feature x∈C_{candidate} that maximizes the AUC among all feature sets C_{candidate}−{x}. If the removal of x improves the highest AUC obtained by C_{candidate}, x is deleted from C_{candidate}. As long as this removal improves upon the highest AUC obtained so far, the removal step is repeated. SFFS repeats the addition and removal steps until AUC reaches 1.0 or the number of additions and deletions exceeds 20, and the feature set with the highest AUC thus far is chosen as the optimal feature set. The classification capability of a feature set, required for feature selection, is measured by the area under the ROC curve (AUC), obtained by cross-validation on the training set.

### 5. Classification

There are two levels of classification. In the first, IR spectral data is used to provide histologic images where each pixel has been classified as a cell type. In the second, the measures from H&E images and IR images are used to classify tissue into disease states. The first classification task is not discussed herein as its development and results are well-documented [35]. For the latter task, a classification algorithm, support vector machine (SVM) [36] was used. Two cost factors are introduced to deal with an imbalance in training data [37]. The ratio between two cost functions was chosen as

$$\frac{{C}_{+}}{{C}_{-}} = \frac{number of negative training examples}{number of positive training examples}$$

to make the potential total cost of the false positives and the false negatives the same.

Additional information on the SVM method is provided below.

Given input data with two classes (+1, −1), SVM [36] constructs a separating hyperplane which aims at maximizing the margin between two classes. Constructing the hyperplane is equivalent to minimizing the structural risk function given by

$$V ( ω , b , ξ ) = \frac{1}{2} {ω}^{T} ω + C {\sum }_{i = 1}^{n} {ξ}_{i}$$

$$subject to \text{:} {y}_{i} [ ω {x}_{i} + b ] \geq 1 - {ξ}_{i} and {ξ}_{i} > 0 , i = 1 , … , n$$

where C is a parameter controlling tradeoff between training error and model complexity, yᵢ is a class label, ξᵢ is slack variable, and n is the number of training examples. It is known that the dual representation of the above problem is easier to solve and given by

$$minimize \text{:} W ( α ) = - {\sum }_{i = 1}^{n} {α}_{i} + \frac{1}{2} {\sum }_{i = 1}^{n} {\sum }_{j = 1}^{n} {y}_{i} {y}_{j} {α}_{i} {α}_{j} ( {x}_{i} \cdot {x}_{j} )$$

$$subject to \text{:} {\sum }_{i = 1}^{n} {y}_{i} {α}_{i} and 0 \leq {α}_{i} \leq C , i = 1 , … , n$$

where αᵢ is a Lagrange multiplier. SVM was originally proposed as a linear classifier, but it could learn a non-linear classifier by replacing the inner-products (xᵢ·xᵢ) by a kernel function K(xᵢ, xⱼ). In this study, we use the Radial basis kernel K(xᵢ, xⱼ)=exp(−γ∥xᵢ−xⱼ∥²) with γ=10, 1, 0.1, 0.01, 0.001.

An imbalance of positive and negative samples in training data may cause the hyperplane computed by SVM to be biased toward either of two classes. To deal with this problem, different cost factors C₊ and C₋ are often introduced in the structural risk function to adjust the cost of false positives and false negatives, and the problem becomes [37]

$$minimize \text{:} V ( ω , b , ξ ) = \frac{1}{2} {ω}^{T} ω + {C}_{+} {\sum }_{i : {y}_{i} = 1}^{n} {ξ}_{i} + {C}_{-} {\sum }_{j : {y}_{j} = - 1}^{n} {ξ}_{j}$$

$$subject to \text{:} {y}_{i} [ ω {x}_{i} + b ] \geq 1 - {ξ}_{i} and {ξ}_{i} > 0 , i - 1 , … , n$$

### 6. Data Preparation

All of the H&E stained images were acquired on a standard optical microscope at 40× magnification. The size of each pixel is 0.9636 um×0.9636 um. On the other hand, the pixel size of IR images is 6.25 um×6.25 um. The acquisition details for the data are provided in [23]. Two data sets, stained under different conditions, were used in this study. The first dataset (“Data1”) consists of 66 benign samples and 115 cancer samples, and the second set (“Data2”) includes 14 benign and 36 cancer samples.

### EXAMPLE 2

### Results

An area under the curve (AUC) value >0.97 was achieved on two data sets that were stained under different conditions. As the classifier trained on one data set and tested on the other data set, ˜0.95 AUC was observed. In the absence of IR data, the performance of the same classification system dropped for both data sets and between data sets.

In summary, a very effective fusion of information from two different microscopy modalities that provide very different types of data with different characteristics was obtained. The method is transparent to a user and does not involve adjustment or decision-making based on spectral data. Combining the IR and optical data achieves the high accuracy values observed in automated detection of prostate cancer in biopsies.

### 1. The Classification System Achieves AUC Greater Than 0.97 on Both Data Sets

K-fold cross validation was performed on each dataset. The data set was divided into K roughly equal-sized partitions, one partition was left out as the “test data”, the classifier was trained on the union of the remaining K−1 partitions (the “training data”) and evaluated on the test data. This was repeated K times, with different choices of the left-out partition (here K=10). In each repetition, cross-validation on the training data was used to select the feature set with the highest AUC as explained in Example 1. The correct and incorrect predictions in the test data, across all K repetitions, were summarized into a ROC plot and the AUC was computed, along with specificities when sensitivity equals 90, 95, or 99%. Since the cross-validation exercise makes random choices in partitioning the data set, we examined averages of these performance metrics over 10 repeats of the entire cross validation pipeline. The average AUC for Data1 and Data2 were 0.982 and 0.974 respectively (Table 2, “feature extraction”=“IR & HE”). At 90%, 95%, and 99% sensitivities, the average specificity achieved on Data1 was 94.76%, 90.91%, and 77.80% respectively, while that on Data2 was 92.53%, 84.19%, and 49.54% respectively.

TABLE 2
Classification results via cross-validation.*

| Data- | Feature | AUC | | Sensitivity | Specificity (%) | | |
|---|---|---|---|---|---|---|---|
| set | Extraction | AVG | STD | (%) | AVG | STD | M_{f} |
| Data1 | IR & HE | 0.982 | 0.0030 | 90 | 94.76 | 1.64 | 13 |
| | | | | 95 | 90.91 | 1.62 | |
| | | | | 99 | 77.80 | 5.52 | |
| | HE only | 0.968 | 0.0052 | 90 | 91.64 | 2.26 | 11 |
| | | | | 95 | 83.90 | 1.91 | |
| | | | | 99 | 53.43 | 13.65 | |
| Data2 | IR & HE | 0.974 | 0.0145 | 90 | 92.53 | 7.11 | 7 |
| | | | | 95 | 84.19 | 10.84 | |
| | | | | 99 | 49.54 | 22.51 | |
| | HE only | 0.880 | 0.0175 | 90 | 61.34 | 10.31 | 8 |
| | | | | 95 | 22.21 | 10.06 | |
| | | | | 99 | 11.21 | 6.01 | |

*AVG and STD denote average and standard deviation across ten repeats of cross-validation.
M_{f} is the median size of the feature set obtained by feature selection from training data.
Column “Feature Extraction” indicates if features were obtained using H&E as well as IR data, or with H&E data alone.

One way to interpret the above values is to examine our automated pipeline as a pre-screening mechanism to identify the samples to be examined by a human pathologist. At a “true positive rate” of 99% (which means that only 1% of the cancer samples will be missed by the screen), the “false positive rate” is 22.2% (i.e., 22.2% of the benign samples will make it through the screen) on average for Data1 (Table1), thereby reducing the workload of the pathologist by 4.5-fold. While the error rate of manual pathology determinations is generally accepted to be in 1-5% range, inclusion of confounding cancer mimickers raises the rate to as high as 7.5% [38]. Also noteworthy is the observation that the same algorithm performs consistently well on both data sets, that were obtained from different staining conditions. This speaks to the robustness of the classification framework.

### 2. Classification System is Robust to Staining Conditions

A classifier trained on Data1 had its performance tested on Data2. An average AUC of 0.956 was observed, with average specificity of 88.57%, 81.92%, and 26.86% at sensitivity equaling 90%, 95%, and 99% respectively (Table 3, “feature extraction”=“IR & HE”). These values are competitive with the cross-validation results on Data2 (Table 2), where the training and testing were both performed on (disjoint parts of) Data2.

TABLE 3
Validation between datasets.*

| Feature | | AUC | | Sensitivity | Specificity (%) | | |
|---|---|---|---|---|---|---|---|
| Extraction | Dataset | AVG | STD | (%) | AVG | STD | M_{f} |
| IR & HE | Train | 0.994 | 0.0006 | 90 | 98.30 | 0.68 | 13 |
| | | | | 95 | 96.58 | 1.10 | |
| | | | | 99 | 91.55 | 2.55 | |
| | Test | 0.956 | 0.0089 | 90 | 88.57 | 5.96 | |
| | | | | 95 | 81.92 | 5.28 | |
| | | | | 99 | 26.86 | 15.50 | |
| HE only | Train | 0.986 | 0.0021 | 90 | 97.77 | 0.97 | 10 |
| | | | | 95 | 91.56 | 2.49 | |
| | | | | 99 | 79.29 | 4.47 | |
| | Test | 0.918 | 0.0100 | 90 | 65.51 | 8.37 | |
| | | | | 95 | 46.14 | 7.53 | |
| | | | | 99 | 13.29 | 6.94 | |

*A classifier is trained on Data1 and tested on Data2.
AVG and STD denote the average and standard deviation.
M_{f} is the median size of the optimal feature set.
Column “Feature Extraction” indicates if features were obtained using H&E as well as IR data, or with H&E data alone.
Column “Dataset” indicates if the performance metrics are from training data (Data1) or from test data (Data2).

### 3. Role of IR Data to Classification Performance

To assess the utility of the IR-based cell-type classification, the above exercises were repeated after extracting features without the guidance of the IR data; i.e., epithelial cells were predicted from the H&E images alone. All of the features defined in §3 were used, except for “Spatial association of lumens and apical regions”, since the distinction between cytoplasm-rich and nuclear-rich region in epithelial cells was unclear in H&E images.

The results from this disadvantaged classifier are shown in Tables 2 and 3 (“feature extraction”=“HE only”). For both types of experiments, lower average AUCs and specificity values were obtained. For instance, the AUC of cross-validation in Data2 (Table 2) dropped from 0.974 to 0.880. Similarly, the results of validation between datasets (Table 3) were substantially worse now compared to the IR-guided classification, with the AUC dropping from 0.956 to 0.918. This indicates that feature extraction with the help of the IR cell-type classification is important to consistent and reliable classification of cancer versus benign tissue samples.

Previously, Tabeshi et al. achieved an accuracy of 96.7% via cross validation in cancer/no-cancer classification [19]. Color, morphometric, and texture features were extracted, and all images were acquired under similar conditions. The disclosed classification result (Table 2), based solely on morphology, is comparable to their result; however the software developed by Tabeshi et al. was not available for evaluation in our data sets. Color and texture features could provide additional information; however, their robustness to different data sets is questionable, and their interpretation is not as obvious as that of morphological features, which are used in clinical practice. Different data sets may have varied properties which may be attributable to staining variations, inconsistent image acquisition settings, and image preparation. The performance of the same method based on texture features has been seen to greatly change from one data set to another [15, 19, 21]. Variations in staining may affect color features. In contrast, morphological features were shown to be robust to varying image acquisition settings [16]. Nonetheless, the quality of morphological features is subject to segmentation of histologic objects. Thus, any method based on morphological features will benefit from the IR cell-type classification.

### 4. Classification Results

The performance of the method was measured by performing 10-fold cross-validation on each dataset and validation between datasets. Each experiment was repeated by using different values of parameter γ for SVM to examine the effect of the value of the parameter. Regardless of the parameter values, high classification performance was achieved in cross-validation of each dataset (Table 4). >0.96 AUCs were achieved for different values of the parameter except the cross-validation on Data2 setting γ=10 (˜0.91 AUC). As a classifier is trained on Data1 and tested on Data2, the classification results were comparable to the cross-validation results on Data2 over different values of the parameter γ (Table 5). Using γ=10, an AUC value of ˜0.84 was achieved which is slightly worse than others (>0.91 AUC). In the opposite experiments, i.e., a classifier is trained on Data2 and test on Data1, AUCs>0.83 were obtained using γ=1, 0.1, 0.01, 0.001, and an AUC value of ˜0.71 was achieved using γ=10. These classification results were substantially worse than the cross-validation results on Data1 (Table 6). However, this may indicate the poor generalizability of the classifier built on Data2 due to the small number of samples and its imbalance.

For the experiments without the guidance of IR data, the results, by and large, were consistent in varying the parameter γ, but significant drop in the AUCs was obtained in comparison with the classification results with the guidance of IR data. In sum, the classification results were not sensitive to the choice of the parameter γ except that the AUCs were dropped when γ=10.

TABLE 4
Classification results varying parameter values via cross-validation.*

| | | Feature | AUC | | Sensitivity | Specificity (%) | | |
|---|---|---|---|---|---|---|---|---|
| γ | Dataset | Extraction | AVG | STD | (%) | AVG | STD | M_{f} |
| 10 | Data1 | IR & HE | 0.967 | 0.0059 | 90 | 88.40 | 3.74 | 9 |
| | | | | | 95 | 80.77 | 5.77 | |
| | | | | | 99 | 62.47 | 6.51 | |
| | | HE only | 0.945 | 0.0058 | 90 | 83.21 | 4.45 | 10 |
| | | | | | 95 | 72.63 | 5.10 | |
| | | | | | 99 | 36.78 | 14.03 | |
| | Data2 | IR & HE | 0.914 | 0.0208 | 90 | 63.14 | 11.26 | 4 |
| | | | | | 95 | 42.43 | 9.71 | |
| | | | | | 99 | 31.34 | 8.92 | |
| | | HE only | 0.735 | 0.0659 | 90 | 30.18 | 9.88 | 8 |
| | | | | | 95 | 15.69 | 8.31 | |
| | | | | | 99 | 5.04 | 3.79 | |
| 0.1 | Data1 | IR & HE | 0.974 | 0.0048 | 90 | 93.98 | 2.18 | 17.5 |
| | | | | | 95 | 86.91 | 3.82 | |
| | | | | | 99 | 68.08 | 8.29 | |
| | | HE only | 0.959 | 0.0043 | 90 | 92.46 | 1.73 | 13 |
| | | | | | 95 | 82.75 | 2.92 | |
| | | | | | 99 | 39.75 | 5.53 | |
| | Data2 | IR & HE | 0.963 | 0.0174 | 90 | 90.48 | 9.46 | 8 |
| | | | | | 95 | 80.40 | 15.38 | |
| | | | | | 99 | 39.59 | 22.07 | |
| | | HE only | 0.901 | 0.0073 | 90 | 70.31 | 9.82 | 12 |
| | | | | | 95 | 33.79 | 13.92 | |
| | | | | | 99 | 15.67 | 12.47 | |
| 0.01 | Data1 | IR & HE | 0.970 | 0.0053 | 90 | 93.47 | 1.66 | 13 |
| | | | | | 95 | 85.50 | 5.77 | |
| | | | | | 99 | 51.31 | 13.05 | |
| | | HE only | 0.955 | 0.0078 | 90 | 90.76 | 2.64 | 12 |
| | | | | | 95 | 78.77 | 3.84 | |
| | | | | | 99 | 28.77 | 11.99 | |
| | Data2 | IR & HE | 0.973 | 0.0160 | 90 | 93.44 | 5.57 | 9 |
| | | | | | 95 | 84.44 | 6.64 | |
| | | | | | 99 | 49.46 | 28.54 | |
| | | HE only | 0.894 | 0.0218 | 90 | 67.57 | 13.59 | 12 |
| | | | | | 95 | 37.36 | 15.81 | |
| | | | | | 99 | 8.87 | 7.1 | |
| 0.001 | Data1 | IR & HE | 0.969 | 0.0074 | 90 | 92.70 | 3.04 | 12 |
| | | | | | 95 | 83.47 | 5.42 | |
| | | | | | 99 | 51.80 | 15.65 | |
| | | HE only | 0.954 | 0.0059 | 90 | 90.62 | 3.59 | 13 |
| | | | | | 95 | 79.19 | 3.77 | |
| | | | | | 99 | 22.41 | 6.84 | |
| | Data2 | IR & HE | 0.967 | 0.0139 | 90 | 92.24 | 3.46 | 10 |
| | | | | | 95 | 85.07 | 6.14 | |
| | | | | | 99 | 40.84 | 24.25 | |
| | | HE only | 0.879 | 0.0186 | 90 | 59.01 | 15.58 | 12 |
| | | | | | 95 | 24.93 | 13.11 | |
| | | | | | 99 | 6.73 | 5.97 | |

*AVG and STD denote average and standard deviation across ten repeats of cross-validation.
M_{f} is the median size of the feature set obtained by feature selection from training data.
Column “Feature Extraction” indicates if features were obtained using H&E as well as IR data, or with H&E data alone.
γ is the parameter of a radial basis kernel for SVM.

TABLE 5
Classification results on Data2 varying parameter values*

| | Feature | | AUC | | Sensitivity | Specificity (%) | | |
|---|---|---|---|---|---|---|---|---|
| γ | Extraction | Dataset | AVG | STD | (%) | AVG | STD | M_{f} |
| 10 | IR & HE | Train | 0.999 | 0.0010 | 90 | 100.00 | 0.00 | 10.5 |
| | | | | | 95 | 99.80 | 0.78 | |
| | | | | | 99 | 97.90 | 2.44 | |
| | | Test | 0.849 | 0.0401 | 90 | 63.60 | 12.20 | |
| | | | | | 95 | 46.90 | 17.55 | |
| | | | | | 99 | 24.29 | 6.99 | |
| | HE only | Train | 0.999 | 0.0003 | 90 | 100.00 | 0.00 | 10.5 |
| | | | | | 95 | 99.85 | 0.33 | |
| | | | | | 99 | 98.84 | 0.90 | |
| | | Test | 0.846 | 0.0442 | 90 | 41.76 | 13.47 | |
| | | | | | 95 | 28.16 | 12.89 | |
| | | | | | 99 | 13.66 | 9.39 | |
| 0.1 | IR & HE | Train | 0.987 | 0.0004 | 90 | 96.13 | 0.60 | 38 |
| | | | | | 95 | 93.77 | 0.76 | |
| | | | | | 99 | 86.11 | 1.39 | |
| | | Test | 0.917 | 0.0069 | 90 | 70.68 | 1.99 | |
| | | | | | 95 | 59.71 | 3.55 | |
| | | | | | 99 | 28.29 | 3.69 | |
| | HE only | Train | 0.979 | 0.0018 | 90 | 97.50 | 1.56 | 14 |
| | | | | | 95 | 91.95 | 2.90 | |
| | | | | | 99 | 52.41 | 13.10 | |
| | | Test | 0.896 | 0.0135 | 90 | 51.90 | 5.89 | |
| | | | | | 95 | 32.46 | 8.99 | |
| | | | | | 99 | 3.16 | 1.85 | |
| 0.01 | IR & HE | Train | 0.984 | 0.0031 | 90 | 96.08 | 1.01 | 34 |
| | | | | | 95 | 94.36 | 2.73 | |
| | | | | | 99 | 80.70 | 6.82 | |
| | | Test | 0.934 | 0.0052 | 90 | 76.48 | 2.62 | |
| | | | | | 95 | 64.29 | 3.01 | |
| | | | | | 99 | 32.57 | 3.01 | |
| | HE only | Train | 0.985 | 0.0225 | 90 | 97.98 | 4.44 | 15 |
| | | | | | 95 | 90.44 | 14.87 | |
| | | | | | 99 | 87.23 | 16.68 | |
| | | Test | 0.893 | 0.0143 | 90 | 53.13 | 17.10 | |
| | | | | | 95 | 25.76 | 6.63 | |
| | | | | | 99 | 8.11 | 5.36 | |
| 0.001 | IR & HE | Train | 0.984 | 0.0032 | 90 | 96.06 | 0.95 | 45 |
| | | | | | 95 | 94.00 | 1.95 | |
| | | | | | 99 | 78.84 | 6.16 | |
| | | Test | 0.937 | 0.0105 | 90 | 78.09 | 5.62 | |
| | | | | | 95 | 65.00 | 6.78 | |
| | | | | | 99 | 32.57 | 3.01 | |
| | HE only | Train | 0.977 | 0.0290 | 90 | 93.85 | 11.40 | 13.5 |
| | | | | | 95 | 83.45 | 24.27 | |
| | | | | | 99 | 81.64 | 27.07 | |
| | | Test | 0.895 | 0.0141 | 90 | 58.81 | 9.71 | |
| | | | | | 95 | 26.07 | 10.47 | |
| | | | | | 99 | 9.33 | 4.58 | |

*A classifier is trained on Data1 and tested on Data2.
AVG and STD denote the average and standard deviation.
M_{f} is the median size of the optimal feature set.
Column “Feature Extraction” indicates if features were obtained using H&E as well as IR data, or with H&E data alone.
Column “Dataset” indicates if the performance metrics are from training data (Data1) or from test data (Data2).
γ is the parameter of a radial basis kernel for SVM.

TABLE 6
Classification results on Data1 varying parameter values*

| | Feature | | AUC | | Sensitivity | Specificity (%) | | |
|---|---|---|---|---|---|---|---|---|
| γ | Extraction | Dataset | AVG | STD | (%) | AVG | STD | M_{f} |
| 1 | IR & HE | Train | 0.998 | 0.0007 | 90 | 100.00 | 0.00 | 9 |
| | | | | | 95 | 99.71 | 0.37 | |
| | | | | | 99 | 95.37 | 1.75 | |
| | | Test | 0.855 | 0.0340 | 90 | 50.18 | 11.52 | |
| | | | | | 95 | 40.41 | 9.88 | |
| | | | | | 99 | 12.33 | 6.29 | |
| | HE only | Train | 0.997 | 0.0050 | 90 | 100.00 | 0.00 | 8 |
| | | | | | 95 | 95.36 | 7.91 | |
| | | | | | 99 | 92.79 | 10.20 | |
| | | Test | 0.804 | 0.0427 | 90 | 48.58 | 10.16 | |
| | | | | | 95 | 37.75 | 9.96 | |
| | | | | | 99 | 22.03 | 9.72 | |
| 10 | IR & HE | Train | 0.998 | 0.0018 | 90 | 99.80 | 0.63 | 7 |
| | | | | | 95 | 99.26 | 1.62 | |
| | | | | | 99 | 97.08 | 4.17 | |
| | | Test | 0.719 | 0.0782 | 90 | 29.41 | 19.22 | |
| | | | | | 95 | 21.83 | 19.42 | |
| | | | | | 99 | 8.12 | 11.02 | |
| | HE only | Train | 0.998 | 0.0018 | 90 | 99.80 | 0.63 | 10 |
| | | | | | 95 | 99.26 | 1.62 | |
| | | | | | 99 | 97.08 | 4.17 | |
| | | Test | 0.773 | 0.0534 | 90 | 39.99 | 10.12 | |
| | | | | | 95 | 24.71 | 11.45 | |
| | | | | | 99 | 12.35 | 7.74 | |
| 0.1 | IR & HE | Train | 0.999 | 0.0009 | 90 | 100.00 | 0.00 | 11.5 |
| | | | | | 95 | 99.71 | 0.90 | |
| | | | | | 99 | 97.09 | 2.56 | |
| | | Test | 0.839 | 0.0287 | 90 | 39.90 | 11.64 | |
| | | | | | 95 | 29.96 | 9.67 | |
| | | | | | 99 | 7.94 | 4.77 | |
| | HE only | Train | 0.988 | 0.0053 | 90 | 100.00 | 0.00 | 9 |
| | | | | | 95 | 93.00 | 5.96 | |
| | | | | | 99 | 69.00 | 12.62 | |
| | | Test | 0.768 | 0.0426 | 90 | 33.06 | 13.28 | |
| | | | | | 95 | 20.51 | 11.69 | |
| | | | | | 99 | 5.05 | 4.79 | |
| 0.01 | IR & HE | Train | 0.999 | 0.0011 | 90 | 100.00 | 0.00 | 13 |
| | | | | | 95 | 99.64 | 0.91 | |
| | | | | | 99 | 97.87 | 2.79 | |
| | | Test | 0.840 | 0.0332 | 90 | 39.77 | 12.94 | |
| | | | | | 95 | 27.99 | 10.46 | |
| | | | | | 99 | 6.02 | 3.08 | |
| | HE only | Train | 0.988 | 0.0042 | 90 | 100.00 | 0.00 | 11 |
| | | | | | 95 | 91.43 | 8.42 | |
| | | | | | 99 | 68.74 | 10.99 | |
| | | Test | 0.773 | 0.0528 | 90 | 30.62 | 16.98 | |
| | | | | | 95 | 15.11 | 13.58 | |
| | | | | | 99 | 2.28 | 2.87 | |
| 0.001 | IR & HE | Train | 0.9999 | 0.0011 | 90 | 100.00 | 0.00 | 13 |
| | | | | | 95 | 99.64 | 0.91 | |
| | | | | | 99 | 97.87 | 2.79 | |
| | | Test | 0.837 | 0.0240 | 90 | 39.46 | 9.58 | |
| | | | | | 95 | 24.86 | 5.27 | |
| | | | | | 99 | 6.59 | 3.93 | |
| | HE only | Train | 0.984 | 0.0066 | 90 | 97.38 | 5.56 | 11 |
| | | | | | 95 | 84.21 | 11.51 | |
| | | | | | 99 | 66.57 | 4.80 | |
| | | Test | 0.769 | 0.0417 | 90 | 29.31 | 13.71 | |
| | | | | | 95 | 14.03 | 8.25 | |
| | | | | | 99 | 3.83 | 5.65 | |

*A classifier is trained on Data2 and tested on Data1 .
AVG and STD denote the average and standard deviation.
M_{f} is the median size of the optimal feature set.
Column “Feature Extraction” indicates if features were obtained using H&E as well as IR data, or with H&E data alone.
Column “Dataset” indicates if the performance metrics are from training data (Data2) or from test data (Data1).
γ is the parameter of a radial basis kernel for SVM.

### 5. Examination of Discriminative Features

The importance of each feature was determined by its rank in the first phase of feature selection, based on its “relevance” to the class label (see above, mRMR). Since different features (e.g., average or standard deviation, global or local features) based on the same underlying quantity (e.g., “lumen roundness”) generally have similar relevance, we examined the average relevance of features in each of 17 feature categories (FIG. 8), for each data set. The complete list of the individual features and their relevance and mRMR rank (for Data1) is available in FIG. 9.

For Data1, lumen-related feature categories are most relevant in general, while epithelium-related feature categories are most important for Data2 (FIG. 8, bars on left). It is surprising that the top 3 feature categories in Data1 (FIG. 8, bars on right)—size of lumen, lumen roundness, and lumen convex hull ratio—have very low relevance in Data2, although that this may be in large part due to variations in staining and malignancy of tumors between the two data sets. Also, examining the features (or feature categories) with highest relevance alone may be slightly misleading, because this examination does not account for redundancy among features.

To further examine the most informative and non-redundant features, the optimal feature sets selected after both stages of the feature selection component were inspected. For Data1, the median size of this set is 13, of which 4 features were always present, i.e., across all repeats of cross validation. These features are number of lumens (L_{MAX}), lumen roundness (G_{AVG}), entropy of nuclei spatial distribution (G_{TOT}) and size of nucleus (G_{TOT}) (FIG. 10). These include both lumen and epithelium related features. Lumen roundness (G_{AVG}) is the only one ranked high by maximal relevance (FIG. 9), yet all four features are consistently chosen by the classifier, since they provide different, complementary information on a tissue: greater circularity of lumens, greater dispersion of nuclei, and increase in the number of lumens and the size of nuclei indicate malignancy of a tissue.

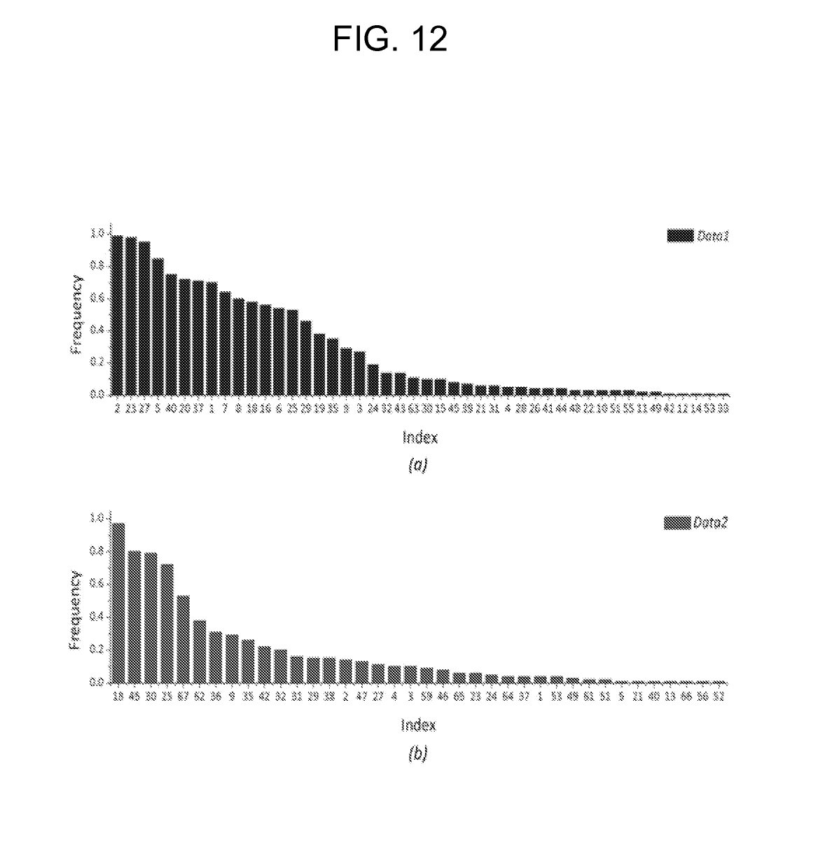

At each iteration of cross-validation, the classifier selects the optimal feature set through two-stage feature selection procedure. It was determined whether the selected features and their importance are consistent over cross-validation or not. As shown in FIGS. 11A and 11B, the maximal relevance of 17 feature categories is consistent within each dataset and over all folds of cross-validation. The features chosen by the classifier are also relatively constant (FIGS. 12A and 12B). The median number of the optimal feature set is 13 and 7 for Data1 and Datat2, respectively. Accordingly, more features have higher frequencies in FIG. 12A.

In summary, the disclosure provides methods to eliminate epithelium recognition deficiencies in classifying H&E images for presence or absence of cancer. The method is entirely transparent to a user and does not involve any adjustment or decision-making based on spectral data. Very effective fusion of the information from two different modalities, namely optical and IR microscopy, that provide very different types of data with different characteristics, was obtained. Several features of the tissue were quantified and employed for classification. Robust classification was achieved using a few measures, which are detailed to arise from epithelial/lumen organization and provide a reasonable explanation for the accuracy of the model. The choice of combining the IR and optical data achieves the high accuracy values observed. The combined use of the two microscopies—structural and chemical—can lead to an accurate, robust and automated method for determining cancer within biopsy specimens.

### REFERENCES

1. Jemal A, Siegel R, Ward E, Murray T, Xu J Q, Smigal C, Thun M J: Cancer statistics, 2006. Ca-Cancer J Clin 2006, 56(2):106-130.
2. Gilbert S M, Cavallo C B, Kahane H, Lowe F C: Evidence suggesting PSA cutpoint of 2.5 ng/mL for prompting prostate biopsy: Review of 36,316 biopsies. Urology 2005, 65(3):549-553.
3. Pinsky P F, Andriole G L, Kramer B S, Hayes R B, Prorok P C, Gohagan J K, P PLCO: Prostate biopsy following a positive screen in the prostate, lung, colorectal and ovarian cancer screening trial. J Urology 2005, 173(3):746-750.
4. Jacobsen S J, Katusic S K, Bergstralh E J, Oesterling J E, Ohrt D, Klee G G, Chute C G, Lieber M M: Incidence of Prostate-Cancer Diagnosis in the Eras before and after Serum Prostate-Specific Antigen Testing. Jama-J Am Med Assoc 1995, 274(18):1445-1449.
5. Pinsky P F, Andriole G L, Kramer B S, Hayes R B, Prorok P C, Gohagan J K, P PLCO: Prostate biopsy following a positive screen in the prostate, lung, colorectal and ovarian cancer screening trial. J Urology 2005, 173(3):746-750 discussion 750-751.
6. Humphrey P A, American Society for Clinical Pathology.: Prostate pathology. Chicago: American Society for Clinical Pathology; 2003.
7. Gleanson D F: Histologic grading and clinical staging of prostate carcinoma. In: The Prostate. Edited by Tannenbaum M. Philadelphia: Lea and Febiger; 1977.
8. Epstein J I, Allsbrook W C, Amin M B, Egevad L L: Update on the Gleason grading system for prostate cancer—Results of an international consensus conference of urologic pathologists. Adv Anat Pathol 2006, 13(1):57-59.
9. Roula M, Diamond J, Bouridane A, Miller P, Amira A: A multispectral computer vision system for automatic grading of prostatic neoplasia. In: Biomedical Imaging, 2002 Proceedings 2002 IEEE International Symposium on: 2002; 2002: 193-196.
10. Diamond J, Anderson N H, Bartels P H, Montironi R, Hamilton P W: The use of morphological characteristics and texture analysis in the identification of tissue composition in prostatic neoplasia. Hum Pathol 2004, 35(9):1121-1131.
11. Stotzka R, Manner R, Bartels P H, Thompson D: A Hybrid Neural and Statistical Classifier System for Histopathologic Grading of Prostatic Lesions. Anal Quant Cytol 1995, 17(3):204-218.
12. Wetzel A W, Crowley R, Kim S, Dawson R, Zheng L, Joo Y M, Yagi Y, Gilbertson J, Gadd C, Deerfield D W et al: Evaluation of prostate tumor grades by content-based image retrieval. In: 1999; Washington, D.C., USA: SPIE; 1999: 244-252.
13. Smith Y, Zajicek G, Werman M, Pizov G, Sherman Y: Similarity measurement method for the classification of architecturally differentiated images. Comput Biomed Res 1999, 32(1):1-12.
14. Jafari-Khouzani K, Soltanian-Zadeh H: Multiwavelet grading of pathological images of prostate. Ieee T Bio-Med Eng 2003, 50(6):697-704.
15. Farjam R, Slotanian-Zadeh H, Zoroofi R A, Khouzani K J: Tree-structured grading of pathological images of prostate. In: Proc SPIE Int Symp Med Imag: 2005; San Diego, Calif.; 2005: 840-851.
16. Farjam R, Soltanian-Zadeh H, Jafari-Khouzani K, Zoroofi R A: An image analysis approach for automatic malignancy determination of prostate pathological images. Cytometry Part B: Clinical Cytometry 2007, 72B(4):227-240.
17. Doyle S, Hwang M, Shah K, Madabhushi A, Feldman M, Tomaszeweski J: Automated grading of prostate cancer using architectural and textural image features. In: Biomedical Imaging: From Nano to Macro, 2007 ISBI 2007 4th IEEE International Symposium on: 2007; 2007: 1284-1287.
18. Naik S, Doyle S, Feldman M, Tomaszewski J, Madabhushi A: Gland Segmentation and Computerized {G}leason Grading of Prostate Histology by Integrating Low-, High-level and Domain Specific Information. In: Proceedings of 2nd Workshop on Microsopic Image Analysis with Applications in Biology, Piscataway, N.J., USA: 2007; 2007.
19. Tabesh A, Teverovskiy M, Pang H Y, Kumar V P, Verbel D, Kotsianti A, Saidi O: Multifeature prostate cancer diagnosis and Gleason grading of histological images. Ieee T Med Imaging 2007, 26(10):1366-1378.
20. Huang P W, Lee C H: Automatic Classification for Pathological Prostate Images Based on Fractal Analysis. Ieee T Med Imaging 2009, 28(7):1037-1050.
21. Farjam R, Soltanian-Zadeh H, Jafari-Khouzani K, Zoroofi R A: An image analysis approach for automatic malignancy determination of prostate pathological images. Cytom Part B-Clin Cy 2007, 72B(4):227-240.
22. Schulte E K W: Standardization of Biological Dyes and Stains—Pitfalls and Possibilities. Histochemistry 1991, 95(4):319-328.
23. Fernandez D C, Bhargava R, Hewitt S M, Levin I W: Infrared spectroscopic imaging for histopathologic recognition. Nat Biotechnol 2005, 23(4):469-474.
24. Ellis D I, Goodacre R: Metabolic fingerprinting in disease diagnosis: biomedical applications of infrared and Raman spectroscopy. Analyst 2006, 131(8):875-885.
25. Spectrochemical Analysis Using Infrared Multichannel Detectors. In: Edited by Rohit Bhargava I W L. Oxford: Blackwell Publishing; 2005: 56-84.
26. Diem M, Chalmers J M, Griffiths P R: Vibrational spectroscopy for medical diagnosis. Chichester, England; Hoboken, N.J.: John Wiley & Sons; 2008.
27. Bhargava R, Hewitt S M, Levin I W: Unrealistic expectations for IR microspectroscopic imaging-Reply. Nat Biotechnol 2007, 25(1):31-33.
28. Brown L G: A Survey of Image Registration Techniques. Comput Surv 1992, 24(4):325-376.
29. Nelder J A, Mead R: A Simplex-Method for Function Minimization. Comput J 1965, 7(4):308-313.
30. Lee J S: Speckle Suppression and Analysis for Synthetic Aperture Radar Images. Opt Eng 1986, 25(5):636-643.
31. Pizer S M, Amburn E P, Austin J D, Cromartie R, Geselowitz A, Greer T, Terhaarromeny B, Zimmerman J B, Zuiderveld K: Adaptive Histogram Equalization and Its Variations. Comput Vision Graph 1987, 39(3):355-368.
32. Dougherty E R: An introduction to morphological image processing. Bellingham, Wash., USA: SPIE Optical Engineering Press; 1992.
33. Peng H C, Long F H, Ding C: Feature selection based on mutual information: Criteria of max-dependency, max-relevance, and min-redundancy. Ieee T Pattern Anal 2005, 27(8):1226-1238.
34. Pudil P, Novovicova J, Kittler J: Floating Search Methods in Feature-Selection. Pattern Recogn Lett 1994, 15(11):1119-1125.
35. Bhargava R: Towards a practical Fourier transform infrared chemical imaging protocol for cancer histopathology. Anal Bioanal Chem 2007, 389(4):1155-1169.
36. Vapnik V N: The nature of statistical learning theory. New York: Springer; 1995.
37. Morik K, Brockhausen P, Joachims T: Combining Statistical Learning with a Knowledge-Based Approach—A Case Study in Intensive Care Monitoring. In: Proceedings of the Sixteenth International Conference on Machine Learning. Morgan Kaufmann Publishers Inc.; 1999: 268-277.
38. Berney D M, Fisher G, Kattan M W, Oliver R T D, Moller H, Fearn P, Eastham J, Scardino P, Cuzick J, Reuter V E et al: Pitfalls in the diagnosis of prostatic cancer: retrospective review of 1791 cases with clinical outcome. Histopathology 2007, 51(4):452-457.

In view of the many possible embodiments to which the principles of the disclosed invention may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of diagnosing prostate cancer in a subject, comprising:
fixing a prostate sample obtained from the subject;
staining the prostate sample with hematoxylin and eosin;
acquiring a Fourier transform infrared (FT-IR) spectroscopic image of the sample;
acquiring an hematoxylin and eosin image of the sample;
overlapping the Fourier transform infrared (FT-IR) spectroscopic image with the hematoxylin and eosin image, thereby generating an overlapped image;
identifying epithelial cells in the overlapped image;
identifying nuclei and lumens in the epithelial cells in the overlapped image;
extracting and classifying features from the nuclei, epithelial cells, and lumens in the overlapped image wherein the features comprise lumen size and nuclei count;
analyzing the extracted and classified features from the nuclei, epithelial cells, and lumens for prostate cancer, and;
diagnosing prostate cancer in the subject with at least 90% sensitivity in diagnosing prostate cancer versus not prostate cancer and with at least 90% specificity in diagnosing prostate cancer from non-cancerous tissue, wherein smaller lumens and an increase in the number of nuclei relative to a normal prostate control sample indicates that the prostate sample is positive for prostate cancer, and wherein similar lumens and a similar number of nuclei relative to a normal prostate control sample indicates that the prostate sample is negative for prostate cancer, wherein similar is +/−<5%.

2. (canceled)

3. The method of claim 21, wherein the first prostate sample is unstained.

4. The method of claim 21, wherein
the first prostate cancer sample and the second prostate sample are serial tissue sections.

5. The method of claim 1, wherein an increase of at least 25%, at least 50%, at least 75%, or at least 90% in the number of nuclei relative to a normal prostate control sample indicates that the prostate sample is positive for prostate cancer.

6. The method of claim 1, wherein a decrease in lumen volume of at least 25%, at least 50%, at least 75%, or at least 90% relative to a normal prostate control sample indicates that the prostate sample is positive for prostate cancer.

7. The method of claim 1, further comprising treating the subject identified as having prostate cancer.

8. The method of claim 1, further comprising selecting the subject suspected of having prostate cancer and obtaining the prostate sample from the subject.

9. The method of claim 7, wherein the subject is a human subject or mammalian veterinary subject.

10. The method of claim 1, wherein the method has at least 95%, or at least 98% sensitivity in diagnosing prostate cancer versus not prostate cancer.

11. The method of claim 1, wherein the method has at least 95%, or at least 98% specificity in diagnosing prostate cancer from non-cancerous tissue.

12. The method of claim 1, wherein the lumen features further comprise one or more of number of lumens, lumen roundness, lumen distortion, lumen minimum bounding circle ratio, lumen convex hull ratio, symmetric index of lumen boundary, symmetric index of lumen area, and spatial association of lumens and cytoplasm-rich regions.

13. The method of claim 1, wherein the lumen features comprise lumen size, lumen roundness, and lumen convex hull ratio.

14. The method of claim 1, wherein the nuclei features further comprise one or more of size of epithelial cells, size of epithelial nuclei, distance from a nucleus to the closest lumen, distance from a nucleus to the epithelial cell boundary, number of isolated nuclei, number of nuclei distinct from lumens, and entropy of spatial distribution of nuclei.

15. The method of claim 1, wherein the features from the nuclei, epithelial cells, and lumens further comprise:
lumen roundness, lumen convex hull ratio, entropy of nuclei spatial distribution, number of lumens, spatial association of lumen and apical regions, fraction of distant nuclei, lumen minimum bounding circle ratio, size of epithelial cells, lumen distortion, distance to epithelial cell boundary, size of nucleus, number of isolated nuclei, distance to lumen, lumen area symmetry, and lumen boundary symmetry; or
one or more of the 67 features listed in FIG. 9; or
number of lumens, total size of nuclei, lumen roundness, and entropy of nuclei spatial distribution.

16. A non-transitory computer-readable storage medium having computer-executable instructions thereon causing a computer to perform a method of diagnosing prostate cancer, comprising:
acquiring a Fourier transform infrared (FT-IR) spectroscopic image of a first sample and a hematoxylin image of a second sample;
overlaying the hematoxylin image with the FT-IR image;
detecting nuclei and lumen in the overlaid image;
extracting and classifying features of the detected nuclei and lumen; and
analyzing the extracted and classified features for prostate cancer.

17. The non-transitory computer-readable storage medium of claim 16, further including performing image registration on the FT-IR spectroscopic image and the hematoxylin image in order to perform the overlaying.

18. The non-transitory computer-readable storage medium of claim 16, wherein image registration includes converting the images to binary, finding parameters of an affine transformation of the FT-IR spectroscopic image and performing the affine transformation to overlay the images.

19. The non-transitory computer-readable storage medium of claim 16, wherein detecting the nuclei includes performing an adaptive histogram equalization, thresholding an image resulting from the adaptive histogram equalization, performing morphological operations to fill out any missing information from the nuclei and using size, shape and average intensity to identify the nuclei.

20. The non-transitory computer-readable storage medium of claim 16, wherein extracting includes one or more of the following: determining the size of cells, nuclei and lumen; determining distances from the nucleus to the lumen and a cell boundary; determining roundness of the lumen; and determining a number of isolated nuclei and lumen.

21. The method of claim 1, wherein the sample comprises a first sample and a second sample, wherein the first sample is used to obtain the FT-IR spectroscopic image and the second sample is used to obtain the hematoxylin and eosin image.
